(19) United States
(12) Patent Application Publication
(10) Pub. No.: US 2023/0002734 A1
(43) Pub. Date: Jan. 5, 2023

(54) METHODS FOR PRODUCING OR ISOLATING EPICARDIAL CELLS AND USES THEREOF

(71) Applicant: Cambridge Enterprise Limited, Cambridge (GB)

(72) Inventors: Sanjay Sinha, Cambridge (GB); Laure Gambardella, Cambridge (GB)

(21) Appl. No.: 17/755,757

(22) PCT Filed: Nov. 9, 2020
(86) PCT No.: PCT/GB2020/052833

§ 371 Date: May 6, 2022

(30) Foreign Application Priority Data
Nov. 7, 2019 (GB) 1916218.9

(51) Int. Cl. C12N 5/077

(52) U.S. Cl. CPC C12N 5/0656; C12N 5/0657; C12N 2506/45

(57) ABSTRACT

The invention relates to in vitro methods for isolating, or producing selected populations of human epicardial cells derived from human pluripotent stem cells; defined mixtures of said cells, and therapeutic uses thereof. Said population comprises epicardial cells with or without the potential to differentiate into cardiac fibroblasts, or a mixture thereof.

### BACKGROUND TO THE INVENTION

The invention relates to in vitro methods for isolating or producing selected populations of human epicardial cells derived from human pluripotent stem cells, pharmaceutical compositions containing them, and therapeutic uses of such populations.

Despite major advances in the treatment of heart failure due to systolic impairment, therapeutic approaches have fallen short of addressing the cause of the problem; injury of the mammalian heart leads to irreversible loss of contractile myocardial tissue which is incapable of regeneration. At the turn of the millennium heart failure was widely identified as an emerging epidemic. To date 5.6 million patients in the US alone and 23 million worldwide are suffering from heart failure with 50 percent dying within 5 years after being diagnosed. Current treatment is limited to ameliorating symptoms and slowing the natural progression of the disease but fails to compensate for the loss of contractile myocardium post-injury.

The epicardium is an epithelium covering the heart, which is essential for normal cardiac development. During embryonic life, the epicardium provides signals for proliferation, survival, and maturation to the cardiomyocytes. In return, the myocardium provides signals inducing proliferation and epithelial to mesenchymal transition (EMT) in the epicardium. The mesenchymal cells derived from the epicardium (EPDCs) invade the myocardium and become mainly cardiac fibroblasts (CF) and coronary smooth muscle cells (cSMC).

In the human adult, the epicardium is quiescent. It becomes reactivated after ischemic injury but produces EPDCs that are less efficient at migrating and differentiating than their embryonic counterparts. They produce signals that activate the resident cardiac fibroblasts, inducing fibrosis but not myogenesis. Better knowledge of the human epicardium could provide a route to circumvent the regenerative limitations of the human adult heart. Mature cardiac cells in the mammalian heart proliferate very slowly limiting its regenerative capacity after injury. Accordingly, cells dying after infarction are not replaced by new ones but instead characteristic fibrotic scar tissue forms, which interferes with potential regeneration, impairs heart function, and may later result in heart failure. The epicardium is a multipotent cardiovascular progenitor source with therapeutic potential for cardiac fibroblast (CF), smooth muscle cell (SMC), and cardiomyocyte regeneration, due to its integral role in development and its ability to initiate myocardial repair in injured adult tissues.

Human epicardial cells, derived from human pluripotent stem cells, have the ability to differentiate in vitro and produce mature cell types of the heart, notably CFs and SMCs.

In vitro-differentiated human epicardial cells have previously been proposed for use in therapy. In particular, the use of such cells together with human cardiomyocytes as a transplant composition is disclosed in WO2018/170280, incorporated herein by reference. This composition is to be used post-injury to the heart, such as after a myocardial infarction, and assists in regenerating tissues. In this work, it was assumed that the in vitro-differentiated human epicardial cells are a homogenous population of cells, with the ability to form each of the differentiated progeny cells, such as cardiac fibroblasts (CF) and smooth muscle cells (SMC).

Previously, it was assumed that the in vitro-differentiated human epicardial cells were a homogenous population, capable of forming the different mature cells types. However, the inventors have carefully determined that the in vitro-differentiated human epicardial cells are actually a heterogeneous population of cells, with two distinct signatures, which has implications for improving the therapeutic outcome of administering these cells to a damaged heart, and further for drug testing and the like. These improvements include the ability to control the biological outcome in the production of differentiated cell types which in turn allows the ability to control fibrosis, smooth muscle repair and the protection and maturation of cardiomyocytes. Thus providing improved treatments for cardiac injury.

### SUMMARY OF THE INVENTION

The present invention provides methods and compositions comprising substantially pure populations of epicardial cells and mixtures thereof optionally formulated with cardiomyocytes for engraftment and subsequent regeneration of functional heart tissue which find utility in the treatment of heart injury following, for example after a myocardial infarction.

In a first aspect of the invention, there is provided a method for separating in vitro-differentiated human epicardial cells into a first population of cells characterised by higher levels of expression of transcription factor 21 when compared to basonuclin 1, and a second population of cells characterised by higher levels of expression basonuclin 1 when compared to transcription factor 21, the method comprising cell-sorting using a capture agent specific for a cell surface marker for the first population of cells and/or the second population of cells.

The in vitro-differentiated human epicardial cells are obtained from human pluripotent stem cells (human embryonic stem cells or human induced pluripotent stem cells).

Optionally the cell-sorting may be achieved by:
a) Magnetic-activated cell sorting (MACS); or
b) Fluorescence-activated cell sorting (FACS); or
c) Microfluidic cell separation; or
d) Buoyancy-activated cell sorting

Optionally the capture agent may be any binding partner typically an antibody, an antibody fragment such as a domain antibody, a derivative of an antibody, an engineered affinity protein such as affibody, an aptamer (peptide or nucleic acid) or any other antibody mimeticor non antibody capture agent.

In an embodiment of the invention, the cells are separated using high stringency anti-PODXL columns. In another embodiment the cells are separated using high stringency anti-THY1 columns. In a preferred embodiment the cells eluated from the column are utilised as those cells will not have been activated by interaction with the antibody on the column. Utilising a high stringency column in such a way will ensure any cells expressing both TCF21 and PODXL will be retained on the column.

The inventors have discovered through studies described in detail below that in vitro-differentiated human epicardial cells derived from either human induced pluripotent stem cells (hiPSCs) or human embryonic stem cells (hESCs) consist of two major populations. A first population is characterised by higher levels of expression of transcription factor 21 than basonuclin 1 (herein denoted TCF21^{high}) and a second population is characterised by higher levels of expression of basonuclin 1 than transcription factor 21 (herein denoted BNC1^{high}). These differentially expressed genes, which are both transcription factors, characterise the two different cells populations. Transcription factor (TCF21), also known as pod-1, capsulin, or epicardin, is a transcription factor essential for the determination of cardiac fibroblast lineage. It is a basic helix-loop-helix transcription factor very important for heart and kidney development which has been implicated in coronary artery disease and cancer. During heart development, it is particularly involved in the regulation of epicardial function and development of cardiac fibroblasts and coronary smooth muscle cells (Ao, X., Ding, W., Zhang, Y., Ding, D. & Liu, Y. TCF21: a critical transcription factor in health and cancer. Journal of Molecular Medicine (2020) & Hu, H., Lin, S., Wang, S. & Chen, X. The Role of Transcription Factor 21 in Epicardial Cell Differentiation and the Development of Coronary Heart Disease. Frontiers in Cell and Developmental Biology (2020)).

BNC1 is a zinc finger transcription factor. It is highly expressed in epithelia and germ cells of testis and ovary. Some of the BNC1 deficient mice die during embryogenesis for unknown reasons while the surviving ones are overall healthy but sterile with identified abnormalities during corneal wound-healing. BNC1 expression in the epicardium of the heart was first reported by the laboratory of Nadia Rosenthal³⁻⁵. The inventors have shown that BNC1 regulates epicardial heterogeneity and allows the separation of the cells into two populations with different properties. BNC1 is expressed in the human heart during development, and it is suggested as an upstream regulator of a transcriptional hierarchy regulating cell identity. They have also observed the effect of knocking this gene down via gene silencing, since this results in the production of cells of the first population only. The majority of the epicardial cells of both populations have been observed, when exposed to conditions encouraging smooth muscle cell differentiation, to differentiate into smooth muscle cells (hPSC-epi-SMC) to the same extent. In contrast, when exposed to conditions encouraging cardiac fibroblasts, whilst the majority of the epicardial cells of the first population (TCF21^{high} or TCF21⁺) differentiate into cardiac fibroblasts (hPSC-epi-CF), none or extremely few epicardial cells of the second population (BNC1^{high} or BNC1⁺) differentiate into cardiac fibroblasts.

Injections of the whole hPSC-epicardium along with hPSC-derived cardiomyocytes, in comparison with injection of hPSC-derived cardiomyocytes alone in the infarcted rat heart, have demonstrated beneficial effects of the whole hPSC-epicardium on the engraftment and maturation of the hPSC-cardiomyocytes and on stimulating angiogenesis in the endogenous heart tissues (Bargehr et al., Nature Biotechnology, 2019). According to bioinformatic predictions, each population are predicted to have different beneficial effects. The TCF21⁺ population are useful for promoting angiogenesis and promoting vascularisation. The second population would be particular useful for survival and maturation of the hPSC-derived cardiomyocytes. The discovery that the TCF21⁺ population is the only one able to produce fibroblasts, provides an advantage in being able to reduce its quantity when fibrosis needs to be controlled. The capacity of the TCF21⁺ population to promote angiogenesis had been confirmed by in vitro experiments in which the sorted TCF21⁺ cells were able to stabilise longer than the BNC1⁺ ones, an endothelial network generated by HUVECS cells in Matrigel. As a consequence, separating and re-mixing of the two cell types could allow for control of the repair by carefully controlling the proportions of the cell type used.

Thus the second population (BNC1^{high}) of cells or cells enriched with the second population form the basis of a therapeutic method for treating or repairing heart tissue damage preferably without attendant fibrosis. The risk of generating excessive CF in a transplant composition may lead to fibrosis leading to ventricular diastolic dysfunction. Therefore, it is highly desirable to minimise and control the amount of the first population of cells (TCF21^{high}) in any therapeutic application where fibrosis should be minimised. In addition, bioinformatic analysis predicts that the BNC1^{high} cells promote cardiomyocyte function by promoting the maturation, survival, and protection, of the cardiomyocytes.

Nonetheless TCF21^{high} cells find utility in the repair of cardiac tissue where angiogenesis and vascularisation is important. Relatively low levels of TCF21^{high} cells have also been shown to promote the activity of BNC1^{high} cells. As a consequence, mixtures in specific proportions of the two cell types allows for control of the repair by carefully controlling the proportions of the cell type used.

Accordingly the present invention provides a composition of a substantially pure BNC1^{high} cell population. In an alternative embodiment, a composition of a substantially pure TCF21^{high} cell population.

In certain embodiments the compositions of the invention may be provided with human cardiomyocytes. The human cardiomyocytes may be in vitro-differentiated. In one embodiment of this aspect, cells of the invention may be provided in suspension with cardiomyocytes, and the resulting mixture injected directly to the myocardium. In another embodiment of this aspect, a patch comprising human cardiomyocytes that are in vitro-differentiated and the cells of the invention may be grafted directly on to the epicardium.

Conditions encouraging smooth muscle fibre differentiation or cardiac fibroblasts are known to those skilled in the art, and a non-limiting example of conditions includes culturing hPSC-epi cells for about 12 days in CDM-PVA supplemented with PDGF-BB (10 ng/ml, Peprotech) and TGFβ1 (2 ng/ml, Peprotech) to obtain hPSC-epi-SMC and with VEGF-B (50 ng/ml, Peprotech) and FGF-2 (50 ng/ml) to get hPSC-epi-CF (as disclosed in is disclosed in WO2018/170280).

Notably, THY1 (also known as CD90 (Cluster of Differentiation 90), originally discovered as a thymocyte antigen) may be used as a marker for the first and second populations. TCF21^{high} cells, the first population, express THY1 (they are THY1⁺); whilst BNC1^{high} cells (the second population) do not (THY1⁻). The cells may be sorted on this basis, since this is a cell surface marker.

Similarly, PODXL (Podocalyxin Like) may be used as a marker for the first and second populations. TCF21^{high} cells, the first population, do not express PODXL (they are PODXL⁻); whilst BNC1^{high} cells (the second population) do express PODXL (PODXL⁺). The cells may be sorted on this basis, since this is a cell surface marker. In some cases, a small proportion of the in vitro-differentiated epicardial cells may be double-positive, whereby they express both PODXL and THY1. Additionally, a minor population of cells may be double-negative, whereby they do not express either marker.

Thus in a second aspect of the invention, an isolated first population (TCF21^{high}) of in vitro-differentiated human epicardial cells characterised by higher levels of expression of transcription factor 21 when compared to basonuclin 1 is provided. Therefore, a substantially pure population of in vitro differentiated TCF21^{high} cells is provided.

In a third aspect of the invention, an isolated second population (BNC1^{high}) of in vitro-differentiated human epicardial cells characterised by higher levels of expression of basonuclin 1 when compared to transcription factor 21 is provided. Therefore, a substantially pure population of in vitro differentiated BNC1^{high} cells is provided.

As the aforementioned studies detailed below suggest angiogenic potential for the first population (TCF21^{high}) of in vitro-differentiated human epicardial cells, a tailored mixture of first and second populations of in vitro-differentiated human epicardial cells may have therapeutic advantages.

Thus in a fourth aspect of the invention, a mixture of first (TCF21^{high}) and second (BNC1^{high}) populations of in vitro-differentiated human epicardial cells is provided, wherein the first population is characterised by expression of higher levels of transcription factor 21 when compared to basonuclin 1 and the second population is characterised by expression of higher levels of basonuclin 1 when compared to transcription factor 21, wherein the mixture is enriched with either the first or second populations of cells. As used herein, enriched may mean increased to a level above natural or normal levels, found in a heterogeneous population of in vitro-differentiated human epicardial cells.

In a fifth aspect of the invention, an isolated first population (TCF21^{high}) of in vitro-differentiated human epicardial cells according to the second aspect of the invention or an isolated second population (BNC1^{high}) of in vitro-differentiated human epicardial cells according to the third aspect of the invention or a mixture of first and second populations of in vitro-differentiated human epicardial cells according to the fourth aspect of the invention is provided for use as a medicament.

More particularly and in a sixth aspect of the invention, an isolated first population (TCF21^{high}) of in vitro-differentiated human epicardial cells according to the second aspect of the invention or an isolated second population (BNC1^{high}) of in vitro-differentiated human epicardial cells according to the third aspect of the invention or a mixture of first and second populations of in vitro-differentiated human epicardial cells according to the fourth aspect of the invention is provided for use in treating and/or repairing cardiac tissue damage.

Mixtures of the invention can be prepared by simple mixing of the two cell populations in predefined ratios, or one cell type can be added to non-sorted heterogeneous mixtures to enrich for the appropriate cell type.

In a seventh aspect of the invention, an isolated first population (TCF21^{high}) of in vitro-differentiated human epicardial cells according to the second aspect of the invention or a mixture of first and second populations of in vitro-differentiated human epicardial cells according to the fourth aspect of the invention is provided wherein the mixture is enriched with the first population of in vitro-differentiated human epicardial cells for use as cell therapy for cardiac repair, optionally in treating and/or repairing cardiac blood vessel, smooth muscle fibre or cardiac fibroblast damage.

In an eighth aspect of the invention, an isolated second population (BNC1^{high}) of in vitro-differentiated human epicardial cells according to the third aspect of the invention or a mixture of first and second populations of in vitro-differentiated human epicardial cells according to the fourth aspect of the invention is provided wherein the mixture is enriched with the second population of in vitro-differentiated human epicardial cells for use in treating and/or repairing smooth muscle fibre damage, preferably with reduced fibrosis.

In a ninth aspect of the invention, there is provided a method for the production of a first population (TCF21^{high}) of in vitro-differentiated epicardial cells, comprising the knockdown of BNC1 in human pluripotent stem cells. Said knock-down may be performed by introducing methods used to silence genes, such as RNAi (RNA interference), CRISPR, or siRNA (small interfering RNA). Alternatively a small molecule compound capable of knockdown of BNC1 expression or translation may be used. In a similar fashion TCF21 knock down may be prepared.

The cells and mixtures obtainable by the methods of the invention may be put to therapeutic use, for example as a cell therapy, for example in the production of cardiac grafts, or they may be used in vitro as a research tool to assist in the developments of small molecule compounds and other therapeutic agents. Thus the Invention provides a transplant composition comprising the cells and mixtures of the invention.

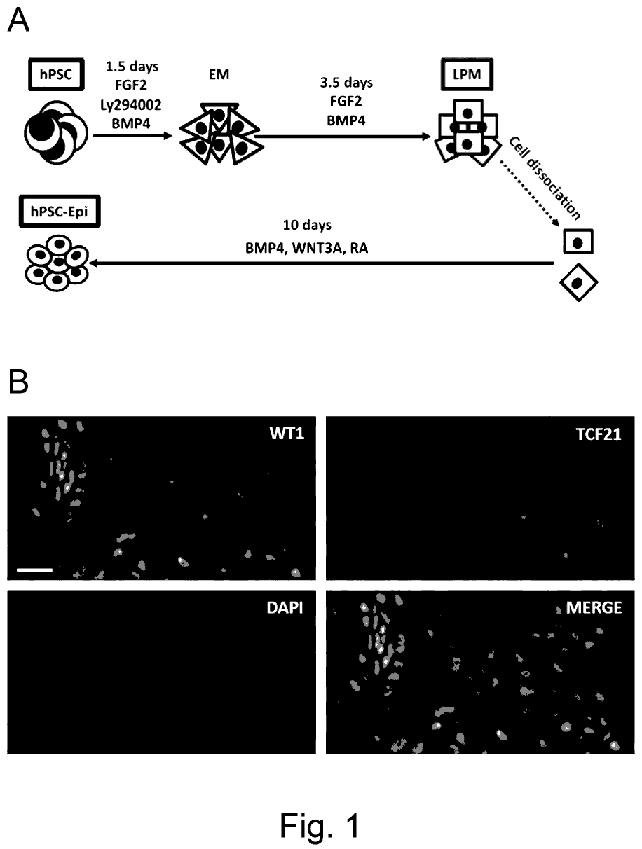

SUMMARY OF THE FIGURES FIG. 1 (A and B): Heterogeneous expression of TCF21 and WT1 in developing human epicardial cells. A) Schematic representation of the hPSC-epi differentiation protocol. EM=Early Mesoderm, LPM=Lateral Plate Mesoderm. RA=Retinoic Acid. B) Detection of WT1 and TCF21 by immunofluorescence in hPSC-epi.

Scale bar=20 μm in B

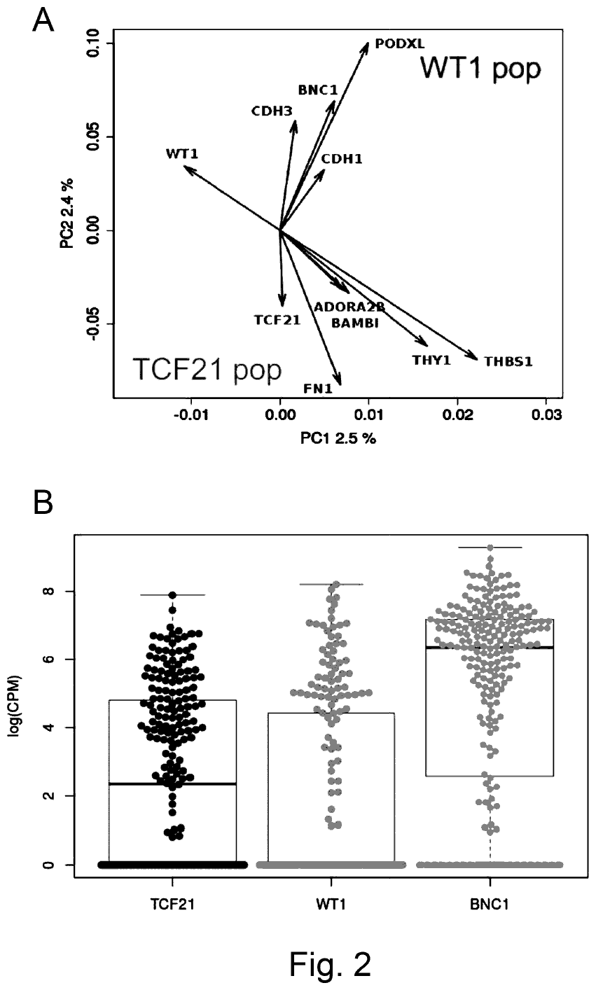

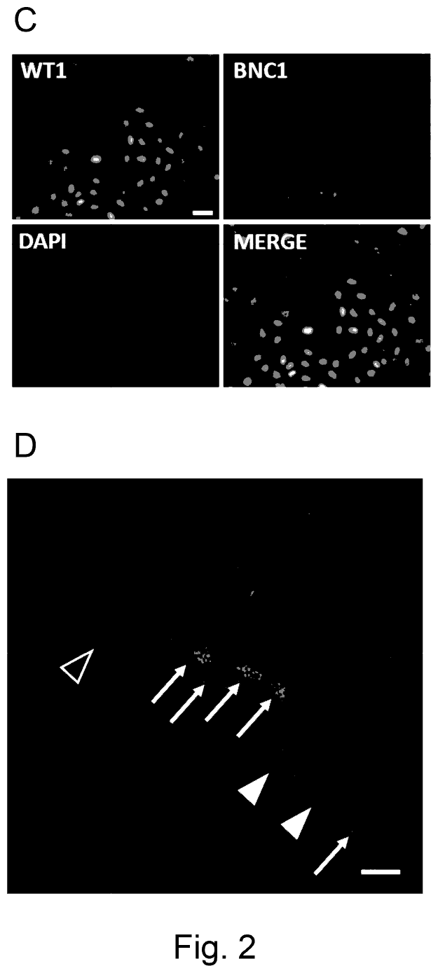

FIG. 2 (A to D): Characterisation of the hPSC-epi heterogeneity by scRNA-seq A) Principal Component Analysis of the gene expression in hPSC-epi cells, showing some of the main gene influences on PC2. B) Distribution of expression of TCF21, WT1 and BNC1 in all epicardial cells (232). The number of cells where no expression is detected are 105, 154 and 44, respectively. C) WT1 and BNC1 detected by immunofluorescence in hPSC-epi cells. D) BNC1 distribution in human epicardium at 8 weeks pc. Arrows point towards high expressing cells, filled arrowheads towards low expressing cells and empty arrowheads to negative cells.

Scale bar=30 μm in D, 9 μm in E and 20 μm in F.

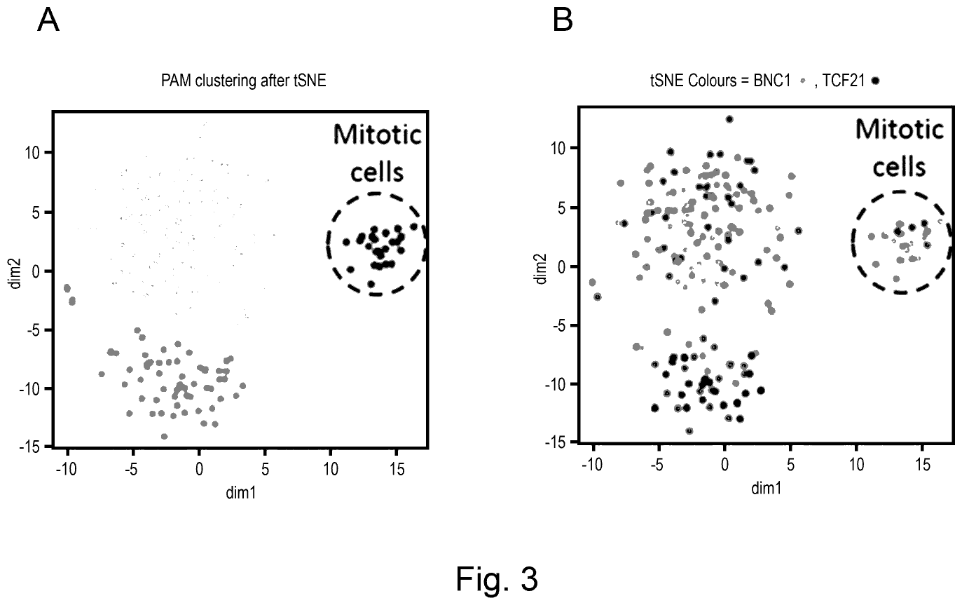

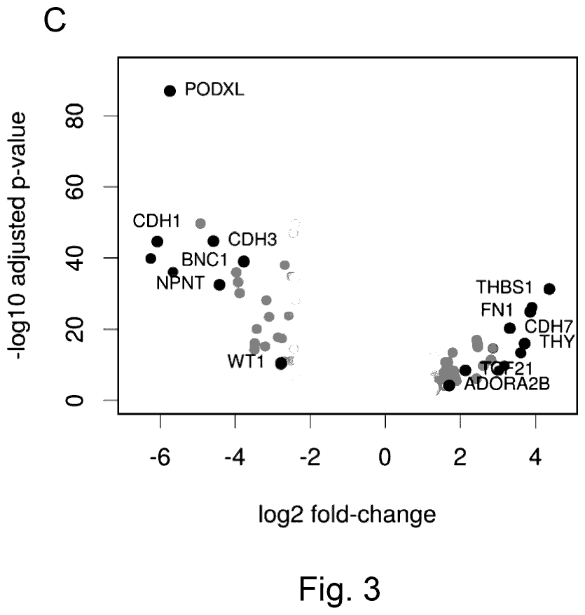

FIG. 3: Transcriptomes of BNC1^{high} and TCF21^{high} sub-populations. A) tSNE of all hPSC-epi cells, followed by a clustering using partition around medoids. B) Expression of TC21 (dark grey) and BNC1 (lighter grey) showing that the main clusters contain either BNC1 or TCF21 cells while the smallest cluster present a mix of them. C) Differential expression analysis between the two main clusters showing the amplitude of changes and their significance. Genes of specific interest for epicardium function or this study are highlighted.

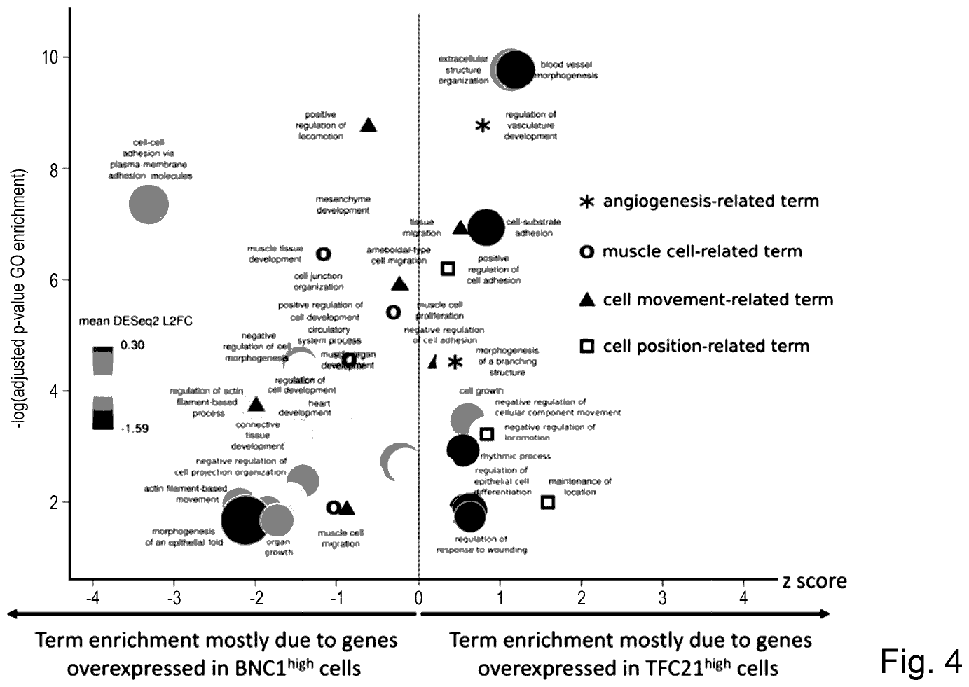

FIG. 4: Predicted tissue and cellular specificities of BNC1^{high} and TCF21^{high} cells. Results of Gene Ontology over-representation and gene expression differential analyses. Each bubble represents an over-represented GO term, the disk size being proportional to the enrichment. The vertical axis presents the significance of the enrichment while the horizontal axis indicates if the term enrichment is mostly due to genes over-expressed in BNC1^{high} cells (negative z-scores) or in TCF21^{high} cells (positive z-scores). Bubble colours show the mean difference of expression, for all the genes annotated by the GO term, between BNC1^{high} cells (turquoise) and TCF21^{high} cells (magenta).

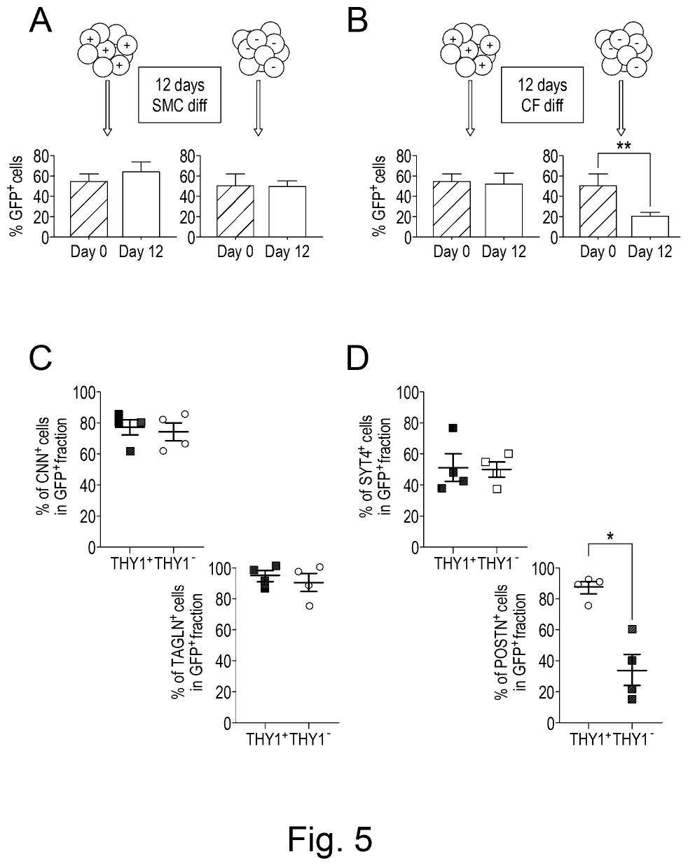

FIG. 5 (A to D): The THY1⁺ population retains the CF potential. THY1 positive (THY1⁺) and THY1 negative (THY1⁻) cells were magnetically separated from a GFP positive (GFP⁺) hPSC⁻epi and mixed with a regular GFP negative hPSC-epi in known proportions measured by flow cytometry. (A) After 12 days of differentiation in SMC medium, the cultures were analysed by flow cytometry to establish the percentage of GFP positive cells still present (A, n=5) and stained for CNN and TAGLN to confirm the differentiation. The % of CNN⁺ or TAGLN⁺ cells present in the GFP⁺ fraction were quantified (C− an average of 31 and 40 GFP⁺ cells from THY1⁺ and THY1- origin were counted in each CNN experiment; an average of 33 and 45 GFP⁺ cells from THY1⁺ and THY1⁻ origin were counted in each sm22α experiment respectively). (B) After 12 days of differentiation in CF medium, the cultures were analysed by flow cytometry to establish the percentage of GFP positive cells still present (A, n=5 and ratio paired t-test performed in Prism 7 from GraphPad) and immunostained for SYT4 and POSTN (b, scale bar=80 μm). The percentage of SYT4 or POSTN positive cells amongst the GFP⁺ population was quantified (d, n=4 and ratio paired t-test performed in Prism 7 from GraphPad. An average of 215 and 67 GFP⁺ cells from THY1⁺ and THY1⁻ origin were counted in each SYT4 experiment. An average of 2125 and 442 GFP⁺ cells from THY1⁺ and THY1⁻ origin were counted in each POSTN experiment).

All displayed error bars are s.e.m.

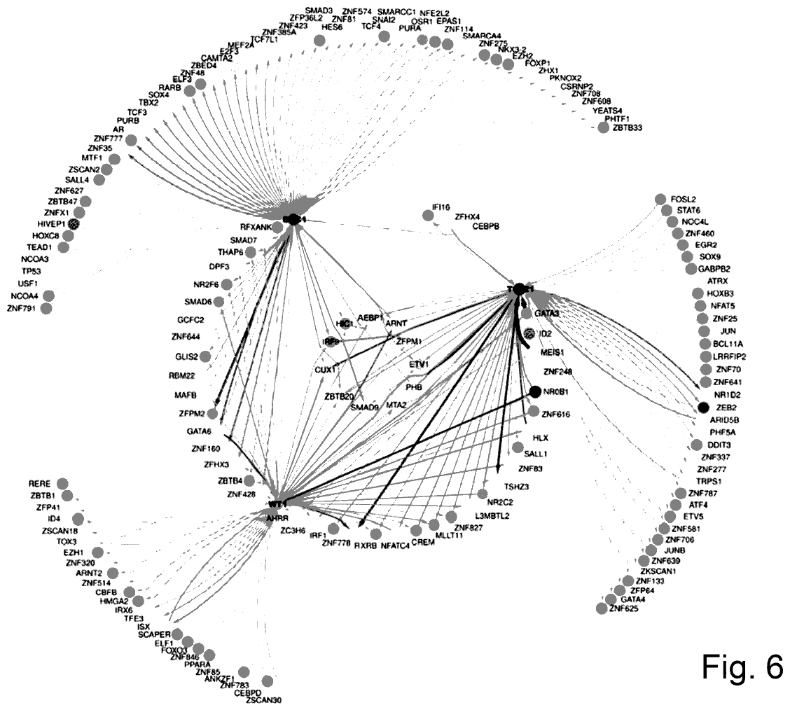

FIG. 6: Core epicardial transcriptional network coordinated by BNC1, TCF21 and WT1. The network is built using the 100 strongest inferred influences between any of BNC1, TCF21 and WT1 and other transcription factors. The central nodes interact with all 3 baits, the nodes on the middle circle interact with 2 of our baits while the nodes on the external circle only interact with one bait. Node colours represent the relative expression of the transcription factor in the two populations, turquoise for BNC1^{high} and magenta for TCF21^{high}. The thickness and density of the edges reflect the likelihood of the inferences. Note that since the network is directed, some pairs of nodes are linked by two edges going in opposite directions, although in most cases only one edge passed our threshold.

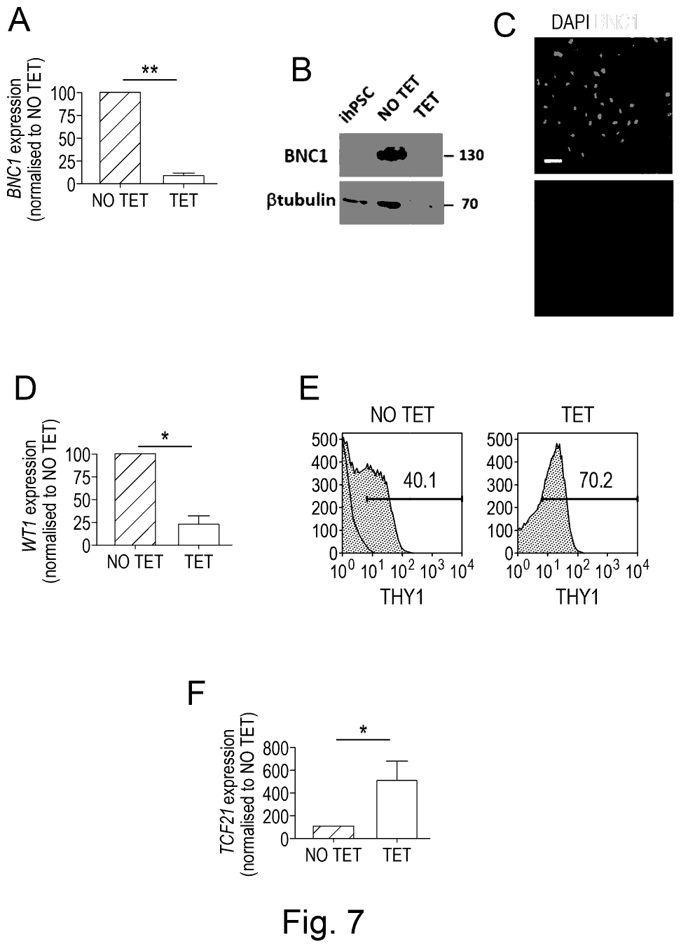

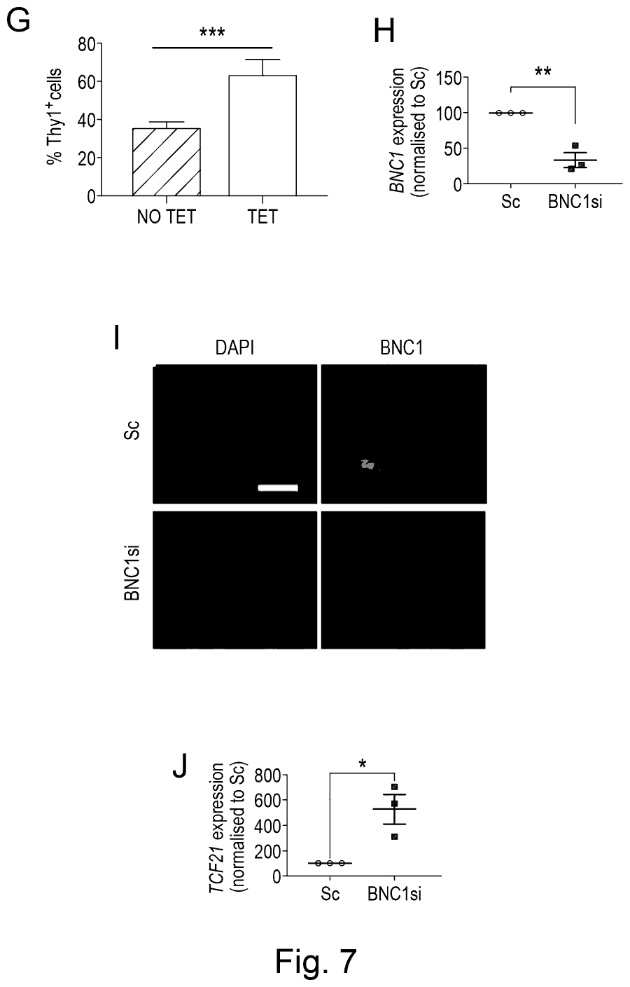

FIG. 7: BNC1 function in developing epicardial cells (a,b,c): hPSC-epi developed from TET-inducible KD hPSC showed 952 (a) more than 90% reduction in BNC1 RNA under TET condition and (b) 98% reduction at the protein level by Western-Blot as (c) also visualised by immunofluorescence (n=5). (D) These cells showed more than 75% reduction of WT1 RNA and (F) a 5-fold increase in TCF21 RNA. (E,G) When BNC1 is silenced during its development, the hPSC-epi is enriched in TCF21^{high} population as revealed by THY1 flow cytometry analysis (histograms of a representative experiment (E) and recapitulative graph (G) of n=5). (H,I,J) BNC1 silencing can be achieved in human foetal primary epicardium using siRNA as shown (H) by RTPCR and (I) immunofluorescence. (J) The KD of BNC1 in human foetal primary epicardium, leads to more than a 5-fold increase in TCF21 RNA n=3. RTPCR data of a, D, F, H and J were obtained by the quantitative relative standard curve protocol as described in Material and Methods. RNA measurements were normalised to housekeeper genes porphobilinogen deaminase (PBGD) or GAPDH.

Scale bar=40 μm in C and D and 20 μm in I.

Statistics were performed with Prism 7 from GraphPad with a ratio paired t-test.

All displayed error bars are s.e.m.

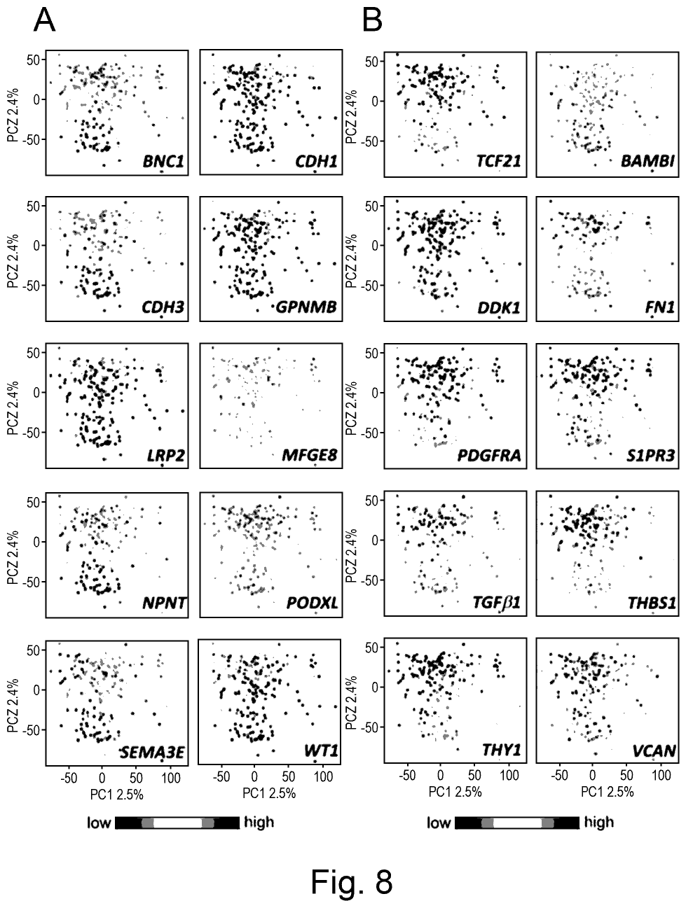

FIG. 8 (A and B): Expression of selected genes characteristic of the two subpopulations. Principal component analysis of the epicardial cells, coloured by the expression of selected genes. A) Genes expressed mainly in BNC1^{high} cells. B) Genes expressed mainly in TCF21^{high} cells.

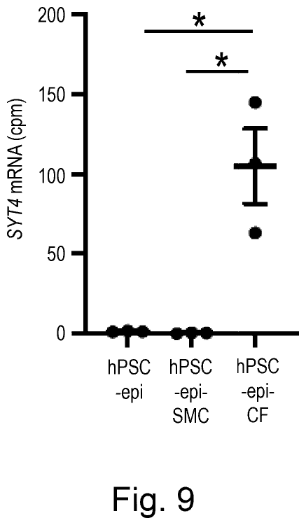

FIG. 9: SYT4 is a marker of hPSC-epi CF. Expression of SYT4 mRNA in counts per million in hPSC-epi, hPSC-epi-SMC and hPSC-epi-CF (n=3 for each type). Error bars are s.e.m. Data were analysed with ratio paired t-test performed in Prism 7 from GraphPad.

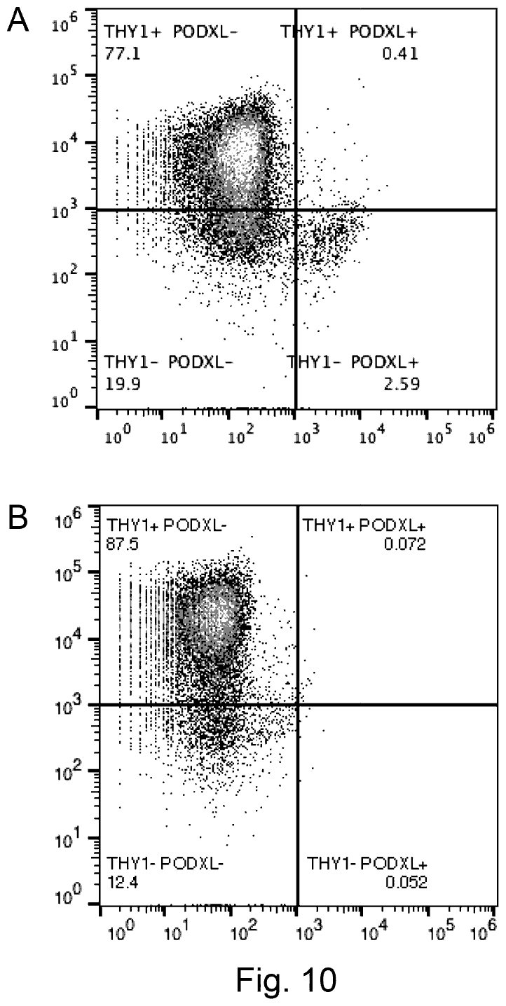

FIG. 10: Sorting of THY1⁺ cells with LD column and anti-PODXL antibody

### DETAILED DESCRIPTION OF THE INVENTION

The present invention is based on the inventors discovery that the in vitro-differentiated human epicardial cells are actually a heterogeneous population of cells, with two distinct signatures and such populations may be separated in to two distinct homogenous poulations. The heart is made of three major tissue layers: the endocardium, myocardium, and epicardium. The epicardium is the outermost epithelial layer of the heart and is responsible for the formation of coronary vascular smooth muscle cells. The epicardium can be re-activated to a more fetal form and/or the epicardial cells can undergo epithelial-to-mesenchymal transition (EMT) in response to an acute injury to the myocardium (e.g., a myocardial infarction).

Provided herein are novel epicardial cell populations and uses thereof in the treatment of cardiac injury, cardiac disease/disorder, and/or promoting vascularization and engraftment of coadministered cardiomyocytes.

In a first aspect of the invention, a method for separating in vitro-differentiated human epicardial cells into a first population of cells characterised by higher levels of expression of transcription factor 21 when compared to basonuclin 1, and a second population of cells characterised by higher levels of expression basonuclin 1 when compared to transcription factor 21, the method comprising cell sorting using a capture agent specific for a cell surface marker for the first population of cells and/or the second population of cells. The populations of cells can be separated based on cell surface markers: TCF21^{high} cells, the first population, express THY1 (they are THY1⁺); whilst BNC1^{high} cells (the second population) do not express this marker (THY1⁻). The cells may be sorted on this basis, since this is a cell surface marker. Other cell markers include PODXL, in this case the first population do not express PODXL (thus PODXL⁻), whilst the second population does express PODXL (PODXL+)

The in vitro-differentiated human epicardial cells are derived from human pluripotent stem cells (hPSCs). These may be autologous stem cells or allogenic stem cells. Optionally the cells are induced pluripotent stem cells (iPSC). Such cells are obtainable from mature cell types using reprogramming methods well known in the art. Alternatively, the use of embryonic stem cells (ESCs) or cell lines derived from ESCs may be possible. The embryonic stem cells may be obtained from a human blastocyst without destruction of said blastocyst. The in vitro-differentiated human epicardial cells may be obtained from the pluripotent stem cells by culturing the cells under the appropriate conditions in order to reach an epicardial fate. The inventors have reported Oyer D, et al. Development. 2015; 142(8):1528-1541, herein incorporated by reference) a method of generating epicardial cells from hPSCs under chemically defined conditions by first inducing an early mesoderm lineage, then lateral plate mesoderm (LM) before further specification to epicardium. They demonstrated that a combination of WNT, BMP and RA signalling promotes robust epicardium differentiation from LM. These in vitro-differentiated human epicardial cells display characteristic epithelial cell morphology and express elevated levels of epicardial markers (such as TBX18, WT1 and TCF21), similar to human foetal epicardial outgrowths. Importantly, these epicardial cells undergo epithelial-to-mesenchymal transition (EMT) and differentiate in vitro into mature and functional SMCs (SMCs), and CFs. hPSCs can be efficiently differentiated to epicardial cells by recapitulating early developmental events in vitro.

As used herein, the term “in vitro-differentiated epicardial cells” refers to epicardial cells that are generated in culture, usually by step-wise differentiation from a precursor such as a stem cell, an early mesoderm cell, a lateral plate mesoderm cell or a cardiac progenitor cell. In one embodiment, the term “in vitro-differentiated epicardial cells” may exclude human tissue-derived epicardial cells obtained from a subject (primary epicardial cells). The term “EMT” or “epithelial to mesenchymal transition” refers to the transition of a cell having an epithelial phenotype to a cell having a mesenchymal phenotype. EMT usually occurs in response to an injury to the myocardium in the adult heart. An epithelial phenotype includes expression of epithelial cell markers (such as cadherin, cytokeratins, ZO-1, laminin, desmoplakin, MUC1). A mesenchymal phenotype includes expression of mesenchymal markers (such as vimentin, fibronectin, twist, FSP-1 Snail, Snai2), with increased cell mobility.

The term “differentiated progeny of epicardial cells” may refer to any of the cells developmentally downstream of, or differentiated from, epicardial cells. Examples of differentiated progeny of most interest in the current invention are smooth muscle cells and cardiac fibroblasts, although epicardial cells may also develop into interstitial fibroblasts, mesenchymal-like cells and possibly endothelial cells, cardiomyocytes or cardiac progenitor cells. The “differentiated progeny of epicardial cells” may refer to any of the differentiated cells that are downstream from any of the epicardial cell populations. As described above, the inventors have determined that BNC1^{high} or TCF21^{high} cells can differentiate into smooth muscle cells, but only TCF21^{high} cells in vitro have been demonstrated to differentiate into cardiac fibroblasts. The smooth muscle cells may be coronary and/or vascular.

The term “marker” may describe a characteristic and/or phenotype of a particular cell. Markers can be used for selection and/or separation of cells comprising characteristics/phenotype of interest. Markers are characteristics, which may be morphological, structural, functional or biochemical characteristics of the cell, or molecules expressed by the cell type. Markers may be cell-surface or intracellular. Markers may be proteins. Such proteins can possess an epitope for capture agents such as antibodies, which allows for the separation of cells based on this marker. Markers may consist of any molecule found in or on a cell, including, but not limited to, proteins (peptides and polypeptides), lipids, polysaccharides, and nucleic acids. The marker may be a cell surface marker, which is preferred for the separation of the cell populations. The marker may be intracellular, such as a transcription factor. If a cell is “positive for” a marker this means that said marker is physically detectable above background levels on the cell using standard methods (e.g. immunofluorescence microscopy or flow cytometry methods, such as fluorescence activated cell sorting (FACS)), or expression of mRNA encoding the marker is detectable above background levels using standard techniques (e.g. RT-PCR). The expression level of a marker may be compared to the expression level obtained from a negative control (cells known to lack the marker). If a cell is “negative for” a marker (alternatively “does not express”) then a marker cannot be detected above background levels on the cell using standard techniques. Alternatively, the terms “negative” or “does not express” means that expression of the mRNA for a marker cannot be detected above background levels using techniques such as RT-PCR. The expression level of a cell surface marker or intracellular marker can be compared to the expression level obtained from a negative control. Thus, a cell that “does not express” a marker appears similar to the negative control with respect to that marker. Relative levels of expression can be determined in a similar fashion, by comparison to a control with a known expression level.

In relation to separating in vitro-differentiated epicardial cells into sub-populations, the presence or absence of a cell surface marker can be used to distinguish the two populations. The cells may be separated using cell-sorting, optionally using cell surface markers that are expressed on one cell population, but not the other. Any suitable method of cell sorting is envisioned. Such methods include, but are not limited to: magnetic-activated cell sorting (MACS), fluorescence-activated cell sorting (FACS), microfluidic cell separation, or buoyancy-activated cell sorting. It is preferred that the cell sorting method relies upon a capture agent. As used herein a “capture agent” may be considered to be an antibody or an antibody mimetic. Optionally the capture agent may be an antibody, an antibody fragment, a derivative of an antibody, a non-antibody protein capture agent such as an affibody, an aptamer (peptide or nucleic acid) or any other antibody mimetic. Those skilled in the art will appreciate that a capture agent will be specific for a target molecule, in relation to the present invention, a marker. The use of a capture agent allows for the selective binding of cells which display said marker, and therefore allows for the cell populations to be separated and/or enriched.

Within the field of cell ontogeny, the term “differentiate/differentiating” is relative and indicates that a “differentiated cell” has progressed further down the developmental pathway than its precursor. The in vitro-differentiated epicardial cells of the invention are therefore further down the developmental pathway than a pluripotent stem cell, but are still capable of further differentiation into mature cell types.

An “isolated cell” is a cell that has been removed from an organism in which it was originally found, or a descendant of such a cell. Optionally the cell has been cultured in vitro. The cells of the invention may be isolated.

As used herein, “substantially pure,” with respect to a particular cell population, refers to a population of cells that is at least about 75%, preferably at least about 85%, more preferably at least about 90%, and more preferably at least about 95% pure, most preferably at least 97% with respect to the cells making up a total cell population. That is, the terms “substantially pure” or “essentially purified”, with regard to a population of BNC1^{high} or TCF21^{high} cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not BNC1^{high} or TCF21^{high} cells, respectively.

As used herein “enriched” may mean that the fraction of cells of one type, such as TCF21^{high}, is increased by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, or by at least 75%, over the fraction of cells of that type in a starting preparation.

Thus a TCF21^{high} enriched population may be at least 40%, by at least 45%, by at least 50%, by at least 55%, by at least 60%, by at least 65%, by at least 70%, or by at least 75%, or by at least 80%, or at least 90% or 95% of the cells in the mixture. The rest of the mixture may comprise BNC1^{high} cells, human cardiomyocytes, or both.

Conversely a BNC1^{high} enriched population may be at least 60%, by at least 65%, by at least 70%, or by at least 75%, or by at least 80%, or at least 90% or 95% of the cells in the mixture. The rest of the mixture may comprise TCF21^{high} cells, human cardiomyocytes, or both.

As used herein “separation” or “selection” refers to isolating different cell types (notably BNC1^{high} or TCF21^{high} cells) into one or more populations and collecting the isolated population as a target cell population which is enriched, for example, in a specific target cell. This can be performed using positive selection, where a target enriched cell population is retained, or negative selection, whereby non-target cell types are discarded.

The cells of the invention may be used to treat conditions involved in cardiac repair. As used herein “treating/treatment” includes reducing or alleviating at least one adverse effect or symptom of a condition, disease or disorder. Treatment may involve administering to a subject an effective amount of a composition, e.g., an effective amount of a cell therapy composition comprising a population of BNC1^{high} or TCF21^{high} cells or a mixture thereof. “Treatment” of a cardiac disorder, a cardiac disease, or a cardiac injury (e.g., myocardial infarction) may be a therapeutic intervention that enhances cardiac function or improves the function of the heart.

In one aspect, the in vitro-differentiated epicardial cells and mixtures thereof, optionally together with cardiomyocytes described herein can be admixed with or grown in or on a preparation that provides a scaffold to support the cells. Such a scaffold can provide a physical advantage in securing the cells in a given location, e.g., after implantation, as well as a biochemical advantage in providing, for example, extracellular cues for the further maturation or, e.g., maintenance of phenotype until the cells are established. Biocompatible synthetic, natural, as well as semi-synthetic polymers, can be used for synthesising polymeric particles that can be used as a scaffold material. In general, for the practice of the methods described herein, it is preferable that a scaffold biodegrades such that the cardiomyocytes and/or epicardial cells can be isolated from the polymer prior to implantation or such that the scaffold degrades over time in a subject and does not require removal. Thus, in one embodiment, the scaffold provides a temporary structure for growth and/or delivery of the cells of the invention or mixtures thereof (optionally with cardiomyocytes) to a subject in need thereof.

In some embodiments, the scaffold permits human cells to be grown in a shape suitable for transplantation or administration into a subject in need thereof, thereby permitting removal of the scaffold prior to implantation and reducing the risk of rejection or allergic response initiated by the scaffold itself.

Preferably the first population (TCF21^{high}) of in vitro-differentiated human epicardial cells expresses at least 5, at least 8, at least 10 times more transcription factor 21 than basonuclin 1.

Preferably the second population (BCN1^{high}) of in vitro-differentiated human epicardial cells expresses at least 5, at least 8, at least 10 times more basonuclin 1 than transcription factor 21.

The cell sorting method may preferably be magnetic-activated cell sorting and fluorescence-activated cell sorting, which are both well-known methods of separating cells for the skilled person in the art. Briefly, in magnetic-activated cell sorting, the cells are treated with magnetic nanoparticles conjugated to antibodies which target specific cell surface proteins. The treated cells are then passed through a column subject to a magnetic field and the cells comprising the specific cell surface proteins are retained in the column and hence are separated from those cells which simply pass through the column. In fluorescence-activated cell sorting, the cells are treated with a fluorescent moiety conjugated to an antibody which targets specific cell surface proteins. The treated cells are entrained in droplets which are then charged with a positive or negative charge depending on whether the cell fluoresces or not. Electrostatic deflection apparatus then diverts each cell into separate pots depending on the charge. Both methods rely on an antibody which targets specific cell surface proteins.

Preferably the cell surface protein marker for the first population (TCF21^{high}) of in vitro-differentiated epicardial cells is selected from the group consisting of THY1, SIPR3, PDGFRA, BAMBI, PLD3, ADAM12, TGFBR3, STRA6, SLC12A8, BEST1, SMIM3, NRP1, ITGA1, TEK, IGDCC4, CD99, ABCA1, CD9, NDRG2, IFITM1 and ACVR2A.

Most preferably the cell surface marker for the first population (TCF21^{high}) of in vitro-differentiated epicardial cells is THY1. This marker is highly expressed, optionally about at least 5, at least 8, at least 10 times higher, or 13 times higher, when compared to the second population (BNC1^{high}) of in vitro-differentiated epicardial cells (FIG. 3C). Thus, THY1 is a useful marker for TCF21^{high} cells. Cells may be separated by using a capture agent specific for THY1, such as an antibody. As used in the Examples, the antibody is preferably a mouse anti-THY1 antibody clone and an anti PODXL antibody clone.

The cell surface protein marker for the second population (BNC1^{high}) of in vitro-differentiated epicardial cells is preferably selected from the group consisting of PODXL, LRP2, ITGA6, TMEM98, CDH3, CDH1, LEPROTL1, SLC34A2, PKHD1L1, AQP1, GPNMB, SLC7A7, CNTN6, CXADR, SLC4A8, PTPRF, ATP7B, ACKR3, SLC2A1, SLC16A3, OLR1, TMEM88, S100A10, CD82, PARM1, PLXNB2 and APLP2.

More preferably the cell surface marker for the second population (BNC1^{high}) of in vitro-differentiated epicardial cells is PODXL. This marker is highly expressed, optionally about at least 5, 10, 15, 20, 25, 30, 35, 40, 45, 50 or 55 times higher, or about 53 times higher, when compared to the first population of cells (FIG. 3C). Cells may therefore be separated using a capture agent specific for PODXL, such as a monoclonal antibody, for Example clone 222328 available from numerous suppliers.

As previously mentioned, it has been observed that under cardiac fibroblast differentiation conditions more cells in the first population (TCF21^{high}) of in vitro-differentiated epicardial cells differentiate forming cardiac fibroblasts than cells in the second population (BNC1^{high}) of in vitro-differentiated epicardial cells. Preferably, more than 50, more than 60, more than 70, more than 80% of the cells in the first population of in vitro-differentiated epicardial cells differentiate forming cardiac fibroblasts. In contrast, it is preferred that less than 50, less than 40, less than 30, less than 20 or less than 10 or 5% of the cells in the second population of human in vitro-differentiated epicardial cells differentiate forming cardiac fibroblasts.

In a second aspect of the invention, an isolated first population (TCF21^{high}) of in vitro-differentiated epicardial cells characterised by higher expression of transcription factor 21 when compared to basonuclin 1 is provided. The in vitro-differentiated epicardial cells are derived from or differentiated from human pluripotent stem cells. Preferably the first population expresses at least 5, at least 8, at least 10 times more transcription factor 21 than basonuclin 1. In addition or alternatively, when under cardiac fibroblast differentiation conditions, preferably more than 50, more than 60, more than 70, more than 80% of the cells in the first population of in vitro-differentiated epicardial cells differentiate forming cardiac fibroblasts.

FIG. 5 demonstrates that 80% of the cells of the first population differentiate into cardiac fibroblasts.

In a third aspect of the invention, an isolated second population (BNC1^{high}) of in vitro-differentiated epicardial cells characterised by higher expression of basonuclin 1 when compared to transcription factor 21 is provided. The in vitro-differentiated epicardial cells are derived from or differentiated from human pluripotent stem cells. When under cardiac fibroblast differentiation conditions, preferably less than 50, less than 40, less than 30, less than 20% of the cells and most preferably less than 10% in the second population of human epicardial cells differentiate forming cardiac fibroblasts.

In a fourth aspect of the invention, a mixture of first (TCF21^{high}) and second (BNC1^{high}) populations of in vitro-differentiated epicardial cells is provided, wherein the first population is characterised by higher levels of expression of transcription factor 21 when compared to basonuclin 1 and the second population is characterised by higher levels of expression of basonuclin 1 when compared to transcription factor 21, wherein the mixture is enriched with either the first or second populations.

Preferably the mixture is formed by the combination of unseparated in vitro-differentiated epicardial cells and either the first or second populations. Alternatively the isolated populations can be mixed in the desired proportions. The first population (TCF21^{high}) of in vitro-differentiated epicardial cells preferably expresses at least 5, at least 8, at least 10 times more transcription factor 21 than basonuclin 1. Alternatively or in addition, when under cardiac fibroblast differentiation conditions, preferably more than 50, more than 60, more than 70, more than 80% of the cells in the first population differentiate forming cardiac fibroblasts. In contrast, when under cardiac fibroblast differentiation conditions, preferably less than 50, less than 40, less than 30, less than 20% of the cells in the second population (BNC1^{high}) of in vitro-differentiated epicardial cells differentiate forming cardiac fibroblasts.

In a fifth aspect of the invention, the isolated first population (TCF21^{high}) of in vitro-differentiated epicardial cells according to the second aspect of the invention or the isolated second population (BNC1^{high}) of in vitro-differentiated epicardial cells according to the third aspect of the invention or the mixture of first and second populations according to the fourth aspect of the invention is provided for use as a medicament.

More particularly and in a sixth aspect of the invention, the isolated first population (TCF21^{high}) of in vitro-differentiated epicardial cells according to the second aspect of the invention or the isolated second population (BNC1^{high}) of in vitro-differentiated epicardial cells according to the third aspect of the invention or the mixture of first and second populations according to the fourth aspect of the invention is provided for use in treating and/or repairing cardiac tissue damage.

In a seventh aspect of the invention, the isolated first population (TCF21^{high}) of in vitro-differentiated epicardial cells according to the second aspect of the invention or the mixture of first and second populations of in vitro-differentiated epicardial cells according to the fourth aspect of the invention is provided wherein the mixture is enriched with the first population of in vitro-differentiated epicardial cells for use in treating and/or repairing cardiac blood vessel, smooth muscle fibre or cardiac fibroblast damage.

In an eighth aspect of the invention, the isolated second population (BNC1^{high}) of in vitro-differentiated epicardial cells according to the third aspect of the invention or the mixture of first and second populations of in vitro-differentiated epicardial cells according to the fourth aspect of the invention is provided wherein the mixture is enriched with the second population of in vitro-differentiated epicardial cells for use in treating and/or repairing smooth muscle fibre damage, preferably with reduced fibrosis.

In one embodiment, the fifth to eighth aspects of the invention are methods of treating a person in need thereof with the isolated first population (TCF21^{high}) of in vitro-differentiated epicardial cells according to the second aspect of the invention or the isolated second population (BNC1^{high}) of in vitro-differentiated epicardial cells according to the third aspect of the invention or the mixture of first and second populations of in vitro-differentiated epicardial cells according to the fourth aspect of the invention.

In another embodiment of the invention, the fifth to eighth aspects of the invention are uses of the first population (TCF21^{high}) of in vitro-differentiated epicardial cells according to the second aspect of the invention or the isolated second population (BNC1^{high}) of in vitro-differentiated epicardial cells according to the third aspect of the invention or the mixture of first and second populations of in vitro-differentiated epicardial cells according to the fourth aspect of the invention for the manufacture of a medicament for the therapeutic application. Such medicaments may include cells on a scaffold to facilitate transfer to a patient in need thereof. The invention will now be described with reference to several non-limiting Examples:

Materials and Methods

Tissue Culture

hPSC-derived cells and separation of the THY1⁺ and THY1⁻ hPSC-epi cells

hPSC (H9 line, Wicell, Madison, Wis.) were maintained as previously described (Iyer et al., 2015) and tested every two months for Mycoplasma contamination. hPSC differentiation was performed in CDM-PVA (Iscove's modified Dulbecco's medium (Gibco) plus Ham's F12 NUT-MIX (Gibco) medium in a 1:1 ratio, supplemented with Glutamax-I, chemically defined lipid concentrate (Life Technologies), transferrin (15 μg/ml, Roche Diagnostics), insulin (7 μg/ml, Roche Diagnostics), monothioglycerol (450 μM, Sigma) and polyvinyl alcohol (PVA, 1 mg/ml, Sigma) on gelatin-coated plates. The cells were first differentiated into early mesoderm with FGF-2 (20 ng/ml), LY294002 (10 μM, Sigma) and BMP4 (10 ng/ml, R&D systems) for 36 h. Then, they were treated with FGF-2 (20 ng/ml) and BMP4 (50 ng/ml) for 3.5 days to generate lateral plate mesoderm. The differentiation of lateral plate mesoderm into epicardium (hPSC-epi) was induced by exposure to Wnt-3A (25 ng/ml, R&D systems), BMP4 (50 ng/ml) and Retinoic Acid (4 μM, Sigma) for 8 to 10 days after dissociation and re-plating of the lateral plate mesoderm cells at a density of 24 000 cells per cm².

In the first set of experiments magnetic separation of the THY1⁺ and THY1⁻ hPSC-epi cells was performed using mouse anti-THY1 antibody clone 5E10 (14-0909-82, Thermofisher) diluted 1 in 100, biotinylated horse anti-mouse IgG antibody from Vector Laboratories (BA-2000) diluted 1 in 500 and MACS Streptavidin MicroBeads from Miltenyi Biotec following their instructions.

In a second purification scheme (see example 8) Magnetic separations of the THY1⁺ or PODXL⁺ hPSC-epi populations were performed using mouse anti-THY1 antibody clone 5E10 (14-0909-82, Thermofisher) or mouse anti-human PODXL (MAB1658, R&D) diluted 1 in 100, with goat anti-mouse microbeads (130-048-402, Miltenyi) and LD columns as described by the manufacturer (Miltenyi, 130-042-901) and the eluted cells utilised for further experiments.

hPSC-epi-SMC and hPSC-epi-CF were derived from hPSC-epi following Iyer et al, 2015. Briefly, after splitting, the hPSC-epi cells were cultured for 12 days in CDM-PVA supplemented with PDGF-BB (10 ng/ml, Peprotech) and TGFβ1 (2 ng/ml, Peprotech) to obtain hPSC-epi-SMC and with VEGFB (50 ng/ml, Peprotech) and FGF-2 (50 ng/ml) to get hPSC-epi-CF.

Primary Human Cultures

Human embryonic and foetal tissues were obtained following therapeutic pregnancy interruptions performed at Cambridge University Hospitals NHS Foundation Trust with ethical approval (East of England Research Ethics Committee) and informed consent in all instances.

For embryonic epicardial explants, 8-week post-conception embryonic hearts were harvested and set up under coverslip on gelatin coated plates (0.1% gelatin for 20 minutes at RT, followed by advanced DMEM F12+10% FBS for storage at 37° C.) and primary epicardium medium [1:1 mixture of Dulbecco's modified Eagle's medium (DMEM, Sigma) and Medium 199 (M199, Sigma) containing 100 U/ml penicillin, 100 μg/ml streptomycin, 10% heat-inactivated foetal bovine serum (FBS, Sigma)]. After a few days, when epicardial cells had started to explant, SB-435142 (Sigma Aldrich), 10 μM final concentration, was added to the medium.

For primary foetal epicardial cultures, the heart was removed from foetuses over 10-weeks post-conception. Several patches of the epicardial layer were peeled off with fine dissecting tweezers and set up to grow in a gelatin-coated 12-well tissue culture plate in primary epicardial medium. After 5 days in a humidified incubator at 37° C. and 5% CO2, the growing cells were dissociated with TrypLE™ Express Enzyme and re-plated in primary epicardial medium supplemented with SB-435142 10 μM final. Cells were maintained in the same conditions and passaged 1:2 when confluent.

For primary human foetal fibroblasts, the foetal hearts were harvested 8-9 weeks post-conception, cut in small pieces and digested with collagenase (collagenase IV, Life Technologies, cat no 17104019) at 0.25% in DPBS, for 30 minutes at 37° C. with occasional resuspension. Digested tissue was smashed through a 40 μm cell strainer, washed twice in DPBS and then incubated a further 10 minutes at 37° C. in TrypLE™ Express Enzyme to get a cell suspension. Cells were seeded at 1.2 10⁷ cells per 75-cm² on gelatin coated plates in DMEM (Sigma) supplemented with 10% FCS (Sigma)+1 ng/ml FGF-2 for 20 min at 37° C. At that stage, only fibroblasts had time to adhere. The medium was refreshed removing all the other cell types.

RNA Sequencing

Single Cell Sequencing

Single cells were sorted by flow cytometry into individual wells of a 96-well plate containing lysis buffer (0.2% (v/v) Triton X-100 and 2 U/μl SUPERaseIn RNase Inhibitor (Invitrogen)) and stored at −80° C. Single-cell libraries for RNA sequencing were prepared using the Smart-seq2 protocol (Picelli et al., 2014), whereby 21 cycles were used for the cDNA library preamplification. Illumina Nextera XT DNA sample preparation kit and Index Kit (Illumina Chesterford UK) was used for cDNA tagmentation and indexing. Library size and quality were checked using Agilent High-Sensitivity DNA chip with Agilent Bioanalyser (Agilent Technologies Stockport UK). The pooled libraries of 96 cells were sequenced at the Babraham Institute sequencing facility on an Illumina HiSeq2500 at 100 bp per read. We used one lane per plate, resulting in 250 000 to 5 800 000 reads per sample. The quality of the raw data were assessed using FastQC [https://www.bioinformatics.babraham.ac.uk/projects/fastqc/] for common issues including low quality of base calling, presence of adaptors among the sequenced reads or any other overrepresented sequences, and abnormal per base nucleotide percentage. FASTQ files were mapped to the H sapiens genome GRCh38 using HISAT2 (Kim et al., 2015). We removed the 22 samples (over 384) for which either most of the reads (above 97%) were mapped to the ERCC spike-in, probably representing empty wells, without cells, or for which less than 80% of reads were in genes, or for which less than 2% genes were detected. This represented 2 to 13 samples per 96 wells plate. Over the remaining 362 cells, 130 were from the lateral plate mesoderm stage (hPSC-LM) and the 232 others from hPSC-epi.

Preliminary analysis using Principal Component Analysis showed that a few cells were isolated, far from most of their grouped siblings. Those cells had less reads than others and a low gene count. We therefore removed 36 cells with less than 500 000 reads, and expressing less than 7 000 genes. The expression of genes was quantified using SeqMonk's RNA-Seq pipeline [https://www.bioinformatics.babraham.ac.uk/projects/seqmonk/]. Raw read counts aligned with all exons were summed up for each gene.

Bulk Sequencing

Total RNA was extracted from cultures using the RNeasy mini from Qiagen. DNA contamination was removed from the samples using the DNA-free™ DNA Removal Kit from Ambion (Thermofisher). cDNA synthesis and IIlumina libraries were performed using SMARTer Stranded Total RNA-Seq Kit v2—Pico Input Mammalian kit from TAKARA. Except otherwise stated, the data from bulk cultures was produced as for the single cell libraries (see above). We sequenced the 21 libraries on two lanes. The two BAM files of each samples were then merged, resulting in 10 million to 25 million reads per sample.

The expression of genes was quantified with SeqMonk's RNA-Seq pipeline using a further DNA contamination correction since a sample exhibited homogeneous read coverage in introns and intergenic regions.

Exploration of Transcriptomes

For all data analysis except differential expression, the counts were corrected for library size (counts per million reads). Genes displaying less than 1 read per million in all samples were discarded, as were genes which expression varied by less than twofold across all samples. Counts were then normalised using the rlog function of the DESeq2 R package. hPCS-epis came from two different experiments, and we found a clear batch effect, while there was none between the cells coming from two different plates sequenced on different lane. We corrected for the culture batch effect with the combat function or the sva R package.

Principal Component Analysis were performed using the prcomp function of the stats R package. t-Distributed Stochastic Neighbor Embedding (t-SNE) was done using the rtsne R package. Parameters were explored systematically, and the better results were obtained with a perplexity of 30, and a maximum of 2000 iterations. Varying the acceleration parameter theta between 0 and 0.5 did not change the results significantly. Clusters were defined by the partitioning around medoids algorithm using the pam function of the cluster R package.

Differential expression analyses were performed with the DESeq2 package. Significance was set at a p-value adjusted for multiple testing of 0.01. Over-representation analyses were performed using Webgestalt and the non-redundant version of Gene Ontology Biological Process branch. The background for DESeq2-related enrichment was the list of all genes expressed in at least one cell. We retained all the terms with a p-value adjusted for multiple testing (FDR) under 0.05. The z-score for each GO term was computed as the difference between the number of genes—annotated with this term—upregulated in TCF21_{high}) and those upregulated in in BNC1^{high} divided by the square root of their sum.

Network Inference

Following the conclusions of the DREAMS Challenges analysis (Marbach, D., Costello, J., Kiffner, R., Vega, N., Prill, R., Camacho, D. & Allison, K. (2012) Wisdom of crowds for robust gene network inference. Nature Methods), we used a combination of methods based on different algorithms to infer regulatory networks. We combined the results of CLR, a mutual-information-based approach providing undirected edges, and GENIE3, a tree-based regression approach providing directed edges. Both methods were the best performers in their category at DREAMS. We applied the two methods to transcription factor gene expression coming from the bulk sequencing samples, using filtered CPM as described above. Since CLR only provides undirected edges while GENIE3 provides directed ones, the results of CLR were all mirrored with an identical score on edges in both directions. Only edges with a positive score in GENIE3's results were used. The intersect between edges present in CLR and GENIE3 results were then ranked according to the product of both algorithms' scores. This only retains edges that have either an extremely high score with one method, or consistent scores with both methods. Subnetworks were extracted using gene lists as seeds, retaining only the first neighbours above a threshold. Visualisation and analysis of the resulting networks was done using Cytoscape.

Inducible Knockdown (psOPTIkd)

Design and Annealing of shRNA Oligonucleotides

Oligonucleotides were designed by using the TRC sequence from Sigma. Hairpin A was selected as a validating hairpin as it was demonstrated to work previously in downregulating B2M expression. The oligonucleotides were annealed according to the protocol supplied by Bertero et al, (Development, 2016, 143:4405-4418) and then ligated into the cut psOPTIkd vector using T4 ligase for two hours at room temperature. The ligation mix was transformed into alpha select competent cells (BioLine) according to manufacturers' directions. The transformations were plated onto LB agar plates containing ampicillin before colony PCR screening of transformants.

Colony PCR screening of transformants

Transformants grown on LB agar plates were picked in the morning after plating for colony PCR. AAVsingiKD forward (CGAACGCTGACGTCATCAACC) and reverse (GGGCTATGAACTAATGACCCCG) primers were used; thermocycling conditions were as follows: 95° C. for five minutes, then 35 cycles of: 95° C. for 30 seconds, 63° C. for 30 s and 72° C. for 1 minute. These PCR reactions were run on a 1.5% agarose gel, with positive colonies running at 520 bp. Positive colonies were mini prepped (Qiagen mini prep kit, used according to manufacturers' directions) before Sanger sequencing through Source BioScience, using the protocol for strong hairpin structures. Miniprepped vectors which showed correct insertion of our shRNA sequence were selected for midiprep (Qiagen) and restriction digest with BamHI to check vector fragment size.

Vector Digestion

Briefly, the psOPTIkd vector (kindly supplied by Ludovic Vallier laboratory) was digested using restriction enzymes BgI II and Sal I (ThermoFisher) in FastDigest buffer (ThermoFisher) for 30 minutes at 37° C. to allow insertion of different shRNA sequences against BNC1. The digested vector product was purified with the QIAquick PCR purification kit (QIAGEN) and run on a 0.8% agarose gel before extraction using the QIAEX II Gel Extraction Kit (QIAGEN).

Gene Targeting by Lipofection

For psOPTiKD of hPSC, AAVS1 targeting was performed by lipofection. hPSCs were transfected 24-48 h following cell passaging with 4 μg of DNA and 10 μl per well of Lipofectamine 2000 in Opti-MEM media (Gibco), according to manufacturer's instructions. Briefly, cells were washed twice in PBS before incubation at room temperature for up to 45 minutes in 1 ml OptiMEM (Gibco). While cells were incubated in OptiMEM, DNA-OptiMEM mixtures were prepared. Mix 1 comprised 4 μg DNA (equally divided between the two AAVS1 ZFN plasmids, a kind gift from Ludovic Vallier laboratory, and our shRNA targeting vector) in 250 μl OptiMEM per well of a six-well plate. Mixture 2 comprised 10 μl lipofectamine in 250 μl OptiMEM per well. Mixtures 1 and 2 were prepared and mixed gently before incubation at room temperature for five minutes. 250 μl of Mixture 2 was then added to 250 μl Mixture 1 before incubation at room temperature for 20 minutes. 500 μl transfection mix of 1:1 Mixture 1:Mixture 2 was added in a drop-wise spiral manner around the well of hPSC. Cells were incubated in transfection mix at 37° C. overnight before washing in CDM-BSA II media the next day approximately 18 hours post-transfection. After 2 days, 1 μg ml⁻¹ of puromycin was added to the CDM-BSA II culture media. Individual hPSC clones were picked and expanded in culture in CDM-BSA II following 7-10 days of puromycin selection.

Genotyping siKD hPSC Clones

Clones from gene targeting were screened by genomic PCR to verify site-specific targeting, determine whether allele targeting was heterozygous or homozygous, and check for off-target integrations of the targeting plasmid. All PCRs were performed using 100 ng of genomic DNA as template in a 25 μl reaction volume using LongAmp Taq DNA Polymerase (NEB) according to manufacturers' instructions, including 2.5% volume dimethyl sulphoxide (DMSO). DNA was extracted using the genomic DNA extraction kit from Sigma Aldrich according to manufacturers' instructions.

Inducible BNC1 Knockdown

One homozygous-targeted clone for each vector transfection was selected for subsequent differentiation into hPSC-epi with or without the addition of 1 μg/ml tetracycline (Sigma) to culture media with the aim of mediating BNC1 knockdown. hPSC-epi was successfully differentiated from each clone in the presence and absence of tetracycline. qPCR analysis indicated that clone 1Ei had a very pronounced reduction in BNC1. Another clone was generated with the vector BNC1-E (1E17) and showed the same level of efficiency at downregulating BNC1.

Retroviral Transduction

Production of the Lentiviral Particles

The lentiviral particle supernatant was obtained from transfection of 293T cells with the lentiviral vector of interest using Mirus TransIT-LT1 transfection reagent and the HIV-1 helper plasmid psPAX2 (Addgene 12260) and HIV-1 envelope plasmid pMD2.G (Addgene 12259).

Production of Fluorescent hPSC Lines

While splitting, the H9 cells were transduced with a lentivirus expressing a EGFP reporter under the control of Ef-1α promoter. We used the lentiviral vector PLVTHM (Addgene #12247).

Immunofluorescence

Primary Antibodies were as Follows:

Unconjugated or Alexa Fluor-488 conjugated Rabbit Anti-WT1 [CAN-R9(IHC)-56-2] (Abcam, ab89901 or ab202635; 1/100); Rabbit anti-BNC1 (Atlas Antibodies, HPA063183; 1/200); Rabbit anti-TCF21 (Atlas Antibodies, HPA013189; 1/100); Mouse Anti-THY1 clone 5E10 (ThermoFisher, 14-0909-82; 1/100); Mouse Anti-CNN1 (Sigma, C2687; 1/1000); Rabbit Anti-periostin (Abcam, ab14041; 1/500); Mouse anti-synaptotagmin 4 (Abcam, ab57473; 1/100)

Cultured Cells

Cells were fixed using 4% PFA, permeabilised and blocked with 0.5% Triton-X100/3% BSA/PBS for 60 min at room temperature. Unless otherwise stated, primary antibody incubations were performed at 4° C. overnight and Alexa Fluor-tagged secondary antibodies (Invitrogen) were applied for 1 hour at room temperature. Nuclei were counterstained with DAPI (10 μg/ml, Sigma).

For the double stain TCF21/WT1 and BNC1/WT1, TCF21 (or BNC1) and WT1 were detected sequentially. Anti-TCF21 or anti-BNC1 were first applied overnight and detected with a Rhodamin-FAB fragment goat anti-Rabbit IgG (H+L) from Abcam during 1 hour at RT. Then the anti-WT1 conjugated to alexa Fluor-488 was incubated for 2 hours at RT. For the double stain THY1/WT1 or THY1/BNC1, the cells were first blocked without permeabilisation and THY1 was first detected with the mouse anti-THY1 followed by incubation with anti-mouse conjugated antibody. The cells were briefly post-fixed in PFA 4% and then permeabilised with 0.5% Triton-X100/3% BSA/PBS before being incubated as normally with anti-WT1 or anti-BNC1.

Images were acquired on a Zeiss LSM 700 confocal microscope and analysed with ImageJ software. For the quantification of the number of GFP⁺ cells also positive for CF or SMC markers, the number of GFP⁺ cells was first measured in the green channel. Then the double positive or double negative (depending on the size of the populations) were counted using the merge images. When few GFP⁺ cells were present in the image, the cells were manually counted using the Analyse ImageJ pluggin called ‘cell counter’ (https://imagej.nih.gov/ij/plugins/cell-counter.html). When too many GFP⁺ cells were present to count manually, we used the function ‘Analyse particles’ after adjustment of the threshold and binary transformation of the image. The double positive or double negative were counted using ‘cell counter’.

Cryostat Sections

Foetal human heart was harvested as described for primary human culture of epicardium. The whole heart was snap-frozen in liquid nitrogen and stored at −80C before sectioning in a cryostat after embedding in OCT. 10 μm thick sections were collected onto SuperfrostPlus slides and stored at −80° C. until staining. Staining was performed as described above.

THY1 Flow Cytometry

Each sample of 10\hat{ }6 cells was divided into two tubes. One tube received a mouse isotype control antibody and the other tube was incubated with the mouse anti-THY1 antibody, clone 5E10 (both at 5 μg/ml final concentration) for 1 hour at RT. After a rinse in 1×PBS, the cells were resuspended in chicken anti-mouse 488 or donkey anti-mouse 647 antibody diluted 1 in 500.

Quantitative Real-Time Polymerase Chain Reaction

Total RNA was extracted using the RNeasy mini kit (Qiagen). cDNA was synthesised from 250 ng RNA using the Maxima First Strand cDNA Synthesis kit (Fermentas). Quantitative real-time polymerase chain reaction (qRT-PCR) reaction mixtures were prepared with SYBR green PCR master mix (Applied Biosystems) and run on the 7500 Fast Real-time PCR system by the quantitative relative standard curve protocol against standards prepared from pooled cDNA from each experiment. Melt curves were checked for each experimental run. CT values were normalised to housekeeper genes porphobilinogen deaminase (PBGD) or GAPDH. Primers were supplied by Sigma Aldrich and sequences were as follows:

| | |
|---|---|
| GAPDH Forward: | AACAGCCTCAAGATCATCAGC; |
| GAPDH Reverse: | GGATGATGTTCTGGAGAGCC; |
| WT1 Forward: | CACAGCACAGGGTACGAGAG; |
| WT1 Reverse: | CAAGAGTCGGGGCTACTCCA; |
| TCF21 Forward: | TCCTGGCTAACGACAAATACGA; |
| TCF21 Reverse: | TTTCCCGGCCACCATAAAGG; |
| BNC1 Forward: | GGCCGAGGCTATCAGCTGTACT; |
| BNC1 Reverse: | GCCTGGGTCCCATAGAGCAT |

Western Blotting

To assess BNC1 levels by immunoblotting, lysate from one confluent well of hpsc-epi cells in a six-well plate was separated by SDS PAGE on an 8% acrylamide gel and transferred overnight onto a PVDF membrane. The protein was detected using a rabbit anti-BNC1 antibody (Atlas antibodies) at 1 in 100 dilution followed by chemiluminescence detection via HRP conjugated secondary antibody, diluted 1 in 10,000 (cat no. 7074S, NEB). Mouse anti-α/βtubulin antibody (Cell Signaling Technology) was used at 1 in 1000 as the housekeeping protein.

### Example 1: Molecular Cell Heterogeneity in hPSC-Epi and Human Foetal Epicardial Explant Culture

First we determined the extent of epicardial marker heterogeneity in hPSC-epi cultures. Since both antibodies suitable for the detection of TCF21 and WT1 in human cells were rabbit in origin, we were previously limited to a flow cytometry strategy in which the presence of double positive cells in the hPSC-epi was indirectly estimated Oyer et al., 2015). In the present study, we differentiated the hPSC-epi according to the protocol previously published (FIG. 1A). Then, we co-immunostained using an anti-TCF21 antibody plus an Alexa-568 secondary with sequential application of an anti-WT1 antibody directly conjugated to Alexa-488. This confirmed a clear heterogeneity in the hPSC-epi (FIG. 1B) with single- and double-positive cells. To validate the in vitro hPSC-derived model we generated explant cultures of primary epicardium from 8 week human foetal hearts; co-immunostaining revealed similar heterogeneity in the foetal explants to that observed in the hPSC-derived cells (data not shown). We then sequenced the transcriptome of the hPSC-epi at single cell resolution in order to characterize precisely the molecular cell heterogeneity of these cells and to determine its physiological regulation and functional relevance.

### Example 2: scRNA-Seq Revealed WT1, TCF21 and BNC1 as Indicators of the hPSC-Epi Functional Heterogeneity

Using a Smart-Seq2 based protocol previously used to analyse mouse embryonic cells (Scialdone et al., 2016), we obtained high quality transcriptomes for a total of 232 hPSC-epi single cells. We examined the variation of TCF21 and WT1 expression in the population using scRNA-seq. As we were using a monolayer of cells obtained from a simple in vitro differentiation protocol, we expected subtle levels of heterogeneity in the sequencing data. Indeed, in a principal component analysis, the first two components only absorbed 2.5 and 2.4% of the variance respectively. Moreover, the subsequent Eigen-values were much smaller, and 195 components were needed to absorb 90% of the variance. The strongest loadings of TCF21 and WT1 were on the second component (PC2). Over-representation analyses using the 100 genes with strongest negative and positive PC2 loadings defined two different molecular signatures on the TCF21 and WT1 sides. Among the top genes on the TCF21 side (FIG. 2A), the strongest is coding for Fibronectin (FN1), with others coding for Thrombospondin (THBS1), THY1, CDH7, BAMBI and Adenosine receptor 2B (ADORA2B) (FIG. 8). On the WT1 side the strongest is coding for the Podocalyxin (PODXL), with others coding for Basonuclin (BNC1, second strongest positive loading on PC2), P-cadherin (CDH3), and E-cadherin (CDH1).

The distribution of expression for TCF21, WT1 and BNC1 was bimodal, with a large population of cells showing no expression at all (106, 154 and 45 cells respectively) (FIG. 2B). It is likely that some of those “zeroes” are dropouts. However, the location of those cells on the PCA plot suggest that they are not randomly distributed and most of them, at least when it comes to TCF21 and BNC1 expression, reflect true subpopulations. Twenty-seven cells (12%) expressed all three markers. Colouring the PCA plot with TCF21, WT1 and BNC1 expression clearly shows two populations segregated along PC2 (Data not shown). With a few exceptions, WT1 expressing cells form a subpopulation of BNC1 expressing cells. Cells strongly expressing BNC1 are mostly devoid of TCF21 expression. Finally, immunofluorescence detection of BNC1 and WT1 confirmed the correlation between high level of WT1 and high level of BNC1 and the inclusion of WT1⁺ cells in the BNC1⁺ population in the hPSC-epi (FIG. 2C). Immuno-staining of foetal human heart sections at 8 weeks pc demonstrated the expression of BNC1 in the epicardium and confirmed a heterogeneous distribution of the protein (FIG. 2D). Immuno-staining of human embryonic epicardial explants from human hearts at 8 weeks pc confirmed co-location of WT1 and BNC1 (Data not shown).

BNC1 and TCF21 are not just markers of two sub-populations; they also reflect the state of the entire transcriptome. We computed the Pearson correlation of BNC1, WT1 and TCF21 expression with all the expressed and variable genes. Comparing these correlations, we observe that if the expression of a gene correlates with TCF21 expression, it does not correlate with BNC1 expression (Pearson correlation of −0.454). On the contrary, if the expression of a gene correlates with that of WT1, it also tends to correlate with BNC1 expression (Pearson correlation of 0.293).

### Example 3: The hPSC-Epi is Composed of a BNC1 and a TCF21 Population

Cell subpopulations of hPSC-epis cannot be solely based on the expression of TCF21 and BNC1 due to dropouts—genes which expression is measured as 0 not because there is no mRNA, but because the mRNA was not reverse-transcribed —. Instead we generated cell similarities using t-SNE, exploring different parameter values. The lowest Kullback-Leibler divergence (a measure of how well the t-SNE distances represents the actual distances in genome space) corresponded with final distributions in 3 groups of cells. We attributed cells to each group using a partition around medoid approach, resulting in three clusters of different sizes (146, 62 and 24 cells, FIG. 3A). The largest cluster expressed considerably more BNC1 than TCF21, the intermediate showing the opposite, while the smallest cluster comprised both types of cells (FIG. 3B). We used DESeq2 to compare gene expression between each pair of clusters. Enrichment analysis on the resulting gene sets showed that the small cluster exhibits a clear signal for mitosis). When we corrected for a cell-cycle component with the single-cell Latent Variable Model before running t-SNE and clustering, the small cluster disappeared, suggesting it was not a true sub-population. To avoid confusion, these 24 mitotic cells were omitted in further analyses.

### Example 4: Molecular Signature of BCN1^{high} and TCF21^{high} Populations

Differential gene expression analysis revealed that 2494 genes were differentially expressed between the largest clusters (FIG. 3A): 1454 higher in the BNC1^{high}, cells and 1040 higher in the TCF21^{high} ones (Data not shown). In addition to an enrichment of 13.6 fold in BNC1 expression, the BNC1^{high} cells exhibited 3.6 times more WT1 than TCF21^{high} cells (FIG. 3C). Genes encoding Podocalyxin, E cadherin and P cadherin were also strongly enriched confirming the positive loading of PCA's component 2. In addition to 4.4 fold more TCF21 expression, the TCF21^{high} cells showed enrichments for the markers found in the negative loading of PCA's component 2. Clustering the cells using 142 strongly expressed (base mean above 100), very significantly (adjusted p-value lower than 10′5) and strongly (enrichment over 2 fold) enriched genes, reproduced the clustering based on the whole genome (Data not shown). This means that the most differentially expressed genes are a good representation of the whole transcriptional landscape, providing confidence that the genes significantly differentially expressed between TCF21^{high} and BNC1^{high} are valid markers to separate the two populations.

The top transcription factors, plasma membrane proteins and secreted factors upregulated in BNC1^{high} and TCF21^{high} populations are listed in Table 1. Some of these genes encode for proteins which had already been flagged in the literature as potential regulators of epicardial function in the embryonic or adult diseased heart. The most overexpressed diffusible factor in BNC1high cells was Nephronectin (NPNT). Nephronectin is the functional ligand of Integrin alpha-8/beta-1, which is overexpressed in the TCF21^{high} population, suggesting cross-talk between the two populations.

TABLE 1
differentially expressed transcription factors, plasma membrane, and secreted
proteins (only the most significant hits with a mean expression above a given level are
displayed, ranked by increasing adjusted p-value).

| | Upregulated in BNClhigh | Upregulated inTCF21high |
|---|---|---|
| Transcription | 113 (BNC1, TOX3, MEOX1,1D4, | 67 (HOPX, ZNF469, NROB1, |
| factors | ID2, NFATC2, ARNT2 SMAD6, | TCF21, TCF19, NR4A1, TBX3, |
| (base mean > 10) | MYCN, WT1, DPF3, ID1, ELF3, | MYBL2, KLF7, AHR, MBNL3, |
| | ID3, MAF, TBX18, ASH2L, | ETS1, ETV4, HIF1A, ZNF564, |
| | SOX6, ZNF415, ZNF624, | TSC22D3, JUNB, MLLT11, |
| | ZFPM2, ZNF20, CUX1, NCOR2 | NFKBIZ, MYC, HOXA13, |
| | . . . ) | HOXB9, ZNF167, CAMTA2 |
| | | . . . ) |
| Plasma membrane | 356 (PODXL, LRP2, ITGA6, | 225 (THYl, S1PR3, PDGFRA, |
| proteins | TMEM98, CDH3, CDH1, | BAMBI, PLD3, ADAM12, |
| (base mean > 50) | LEPROTL1, SLC34A2, | TGFBR3, STRA6, SLC12A8, |
| | PKHD1L1, AQP1,GPNMB, | BEST1, SMIM3, NRP1, ITGA1, |
| | SLC7A7, CNTN6, CXADR, | TEK, IGDCC4, CD99, ABCA1, |
| | SLC4A8, PTPRF, ATP7B, | CD9, NDRG2, IFITM1, |
| | ACKR3, SLC2A1, SLC16A3, | ACVR2A, . . . ) |
| | OLR1,TMEM88, S100A10, | |
| | CD82, PARM1, PLXNB2, APLP2 | |
| | . . . ) | |
| Secreted factors | 229 (SERPINE3, SEMA3E, | 187 (THBSI, TGFBI, FN1, |
| (base mean > 50) | NPNT, EDN3, PLTP, OLFML1, | DKKI, |
| | FRZB, SEMA3C, SBSPON, | FREM1, IGFBP3, COL1A1, |
| | MFGE8, APLP2, GAS6, LAMA5, | MGP, |
| | LAMB1, COL21A1, FBN2, | CXCL14, SERPINE2, VCAN, |
| | LAMC1 . . . ) | LUM, |
| | | COL3A1, MFAP4, CHI3L1, |
| | | ADAM12, COL6A2, PRSS23, |
| | | CCL2 . . . ) |

### Example 5: Gene Ontology Analysis Predicts Different Functions for BNC1^{high} and TCF21^{high} Populations

Gene Ontology (GO) over-representation analyses suggested a different phenotypic signature for each population, favouring migration and muscle differentiation for BNC1^{high} and adhesion/angiogenesis for TCF21^{high}. Using the genes differentially expressed between the two populations we ran GO over-representation analyses using Web Gestalt. FIG. 4 illustrates results of the GO term enrichment, after filtering out the terms related to non-cardiac tissues. FIG. 4 focusses on the terms related to cell and tissue processes. The BNC1^{high} population expressed more genes involved in muscle differentiation, migration and cell-cell interaction. In contrast, the TCF21^{high} was characterised by adhesion with the term ‘Cell substrate-adhesion’ showing high significance and high specificity to this population. Moreover FIG. 4 revealed an angiogenic activity restricted to the TCF21^{high} cells. In particular the GO term ‘blood vessel morphogenesis’ showed high significance, the highest z-score, with genes highly specific to TCF21^{high} population (FIG. 4). Furthermore, a large number of genes involved in VEGF production were expressed specifically in the TCF21^{high} population.

### Example 6: THY1 is a Membrane Marker of the TCF21^{high} Population Enriched in CF Potential

In order to separate the two populations, we searched for specific membrane-associated proteins and cell surface receptors. THY1 was 13 times more expressed in TCF21^{high} cells (Table 1). Immunofluorescence confirmed that the distribution of the protein THY1 was indeed negatively-correlated with WT1 in our system (Data not shown) and to WT1 and BNC1 in primary human foetal epicardial explants (Data not shown). As THY1 had not been reported before in the epicardium, we validated its expression on cryosections of human embryonic hearts at 8 weeks pc. The immunofluorescence confirmed a heterogeneous expression of THY1 in the human developing epicardium (Data not shown). We used an anti-THY1 antibody to magnetically separate the two epicardial populations from constitutive GFP-expressing (GFP+) hPSC-epi and analyse their capacity to respond to differentiation signals. To analyse the developmental potential of each population under normal conditions, we mixed each fraction with non-fractionated GFP-negative (GFP−) hPSC-epi cells in equal proportions and recorded the exact percentage of GFP⁺ cells at DO by flow cytometry. The two mixes made of [unfractionated GFP-hPSC-epi and GFP⁺ THY1⁺hPSC-epi] or [GFP− hPSC-epi and GFP⁺ THY1− hPSC-epi] were separately cultured in differentiation medium for SMC and CF. The SMC and CF differentiation media, established previously (Iyer et al., 2015), were made of CDM supplemented with TGFβ and PDGF-BB or VEGFB and FGF-2 respectively. When subjected to SMC differentiation, the proportion of GFP⁺ cells remained unchanged with both THY1 fractions (FIG. 5A, n=5). We stained the cells for two well characterised markers of the SMC lineage, calponin (CNN) and transgelin (TAGLN). Amongst the GFP⁺ cells, we quantified the % of cells positive for these SMC markers and found similar % for THY1⁺ and THY1⁻ origin, indicating they all differentiated well into SMCs (FIG. 5C). In the CF differentiation medium, the proportion of GFP+ cells remained unchanged in the culture containing the THY1⁺ population but was reduced by more than half (19.5% against 50%) in the condition containing the THY1⁻ population (FIG. 5B, n=5). We concluded that the THY1⁻ hPSC-epi cells did not survive well in response to FGF-2 and VEGFB. Reviewing the molecular signature of THY1⁺ and THY1⁻ populations, which coincided with the TCF21⁺ and TCF21⁻ cells respectively, we noted that NRP1, one of the receptors for VEGFB, is mostly expressed in the THY1⁺ fraction (cf Table 1), which could give an advantage to THY1⁺ cells over the THY1⁻ in CF conditions.

Furthermore, we immunostained the cultures for synaptotagmin 4 (SYT4) and periostin (POSTN) (Data not shown). SYT4 has been identified in our in vitro differentiation system as up-regulated Furthermore, we immunostained the cultures for synaptotagmin 4 (SYT4 and periostin (POSTN) (Data not shown). SYT4 has been identified in our in vitro differentiation system as up-regulated in the hPSC-epi CF compared to the hPSC-epi or hPSC-epi SMC (Data not shown). POSTN is a well-established marker of CF. To assess if the surviving GFP⁺ hPSC-epi cells coming from the THY1⁻ origin could acquire a CF signature, we quantified amongst the GFP⁺ cells, the % of SYT4 or POSTN positive cells. There were equivalent numbers of SYT4⁺ regardless of THY1 status (FIG. 5D). However only 37% of the GFP+ cells expressed POSTN with a THYT origin compared to 90% with a THY1+ origin (FIG. 5D). Thus, those THY1⁻ cells that do survive CF conditions respond poorly to the FGF-2⁺ VEGFB stimulus to turn into CF. Since only 40% of the THY1⁻ cells survived under CF differentiation conditions and only 37% of the survivors expressed POSTN, then in total only 15% of the THY1⁻isolated cells acquired a CF identity versus 90% for THY1⁺. Given that we used positive selection to isolate the TCF21/THY1^{high} population, it is likely that a number of TCF21/THY1^{low} cells will have been present in the so-called THY1⁻ fraction; we hypothesise that the CF cells generated from the THY1⁻fraction originated in fact from those TCF21/THY1^{low} cells. Regardless, we can conclude that the THY1⁺ hPSC-epi had a higher propensity (at least 6 times higher with the current data) to become CF than the THY1⁻ fraction.

### Example 7: A Core Epicardial Transcriptional Network is Coordinated by BNC1, TCF21 and WT1

Network inference methods applied to our system positioned BNC1 as a master regulator, sitting on top of an epicardial regulatory network. To better understand the implications of BNC1, TCF21 and WT1 in the regulation of the epicardial development and function, we inferred a directed transcriptional regulatory network using a combination of methods, CLR and GENIE3, as described in the materials and methods. The variation in the system was generated by using the bulk sequencing transcriptomic data from different stages of cell development including SMC differentiated from hPSC-derived lateral plate mesoderm (pre-epicardial stage in our system), hPSC-epi, hPSC-epi-CF and hPSC-epi-SMC. We retained the top 100 predicted functional interactions between any TF and each of BNC1, TCF21 and WT1. BNC1 and TCF21 shared 3 interactors, BNC1 and WT1 shared 17 interactors, and WT1 and TCF21 shared 21 interactors. 11 TFs are interacting with the three baits. The top 100 influences involving TCF21 showed a balanced picture with 48 influences originating from TCF21, and 52 targeting TCF21, many influences being bidirectional. The image was similar for WT1. On the contrary 68% of influences involving BNC1 originated from this gene (FIG. 6, Table 2), the imbalance being even more striking in the 50 strongest interactions, where only 9 influences targeted BNC1. These findings suggest that BNC1 may be a master regulator of epicardial function.

TABLE 2
top 100 influences between one of TCF21, WT1, BNC1 and any transcription factor (genes
are ranked by decreasing likelihood of influences as computed by the combination of CLR score and
GENIE3 rank).

| | Influencing | Influenced by |
|---|---|---|
| BNC1 | ZFPM2, GATA6, ZNF160, ZNF35, ZNF777, AR, | SMAD9, ZFPM2, ZFPM1, |
| | PURB, TCF3, ZFHX3, TBX2, SOX4, RARB, IRF9, | CEBPB, SMAD6, GATA6, PHB, GCFC2, |
| | ZNF428, ELF3, ZNF48, ZBED4, CAMTA2, E2F3, | ZNF791, NCOA4, NFE2L2, ZNF644, |
| | MEF2A, ARNT, ZFPM1, TCF7L1, ZBTB20, RBM22, | AEBP1, USF1, MTA2, MAFB, TP53, NCOA3, |
| | SMAD9, ZNF385A, | TEAD1, HOXC8, SMARCC1, PURA, HIVEP1, |
| | NR2F6, ETV1, ZNF423, DPF3, ZFP36L2, SMAD3, | SOX4, ZNF114, ZNFXI, ZBED4, ZBTB47, |
| | HES6, ZNF81, PHB, ZNF574, TCF4, SNAI2, | ZNF627, SALL4, ZSCAN2, MTF1 |
| | SMARCC1, WT1, THAP6, PURA, GLIS2, OSR1, | |
| | SMAD7, NFE2L2, EPASI, ZNF114, SMARCA4, | |
| | ZFHX4, ZBTB4, ZNF275, NKX3-2, EZH2, HIC1, | |
| | FOXP1, ZHX1, CUX1, PKNOX2, CSRNP2, ZNF708, | |
| | RFXANK, ZNF608, YEATS4, PHTF1, IFI16, ZBTB33 | |
| WTI | TCF21, ZNF778, ARNT, GATA3, CUX1, ZNF83, | NROB1, GATA6, ZNF778, HLX, TCF21, |
| | ISX, SMAD6, MLLT11, TFE3, AEBP1, SMAD9, | ZNF616, ZNF83, NFATC4, NR2C2, ISX, |
| | IRF9, L3MBTL2, MTA2, IRX6, ZBTB20, NR2F6, | SMAD9, GAT A3, ID2, MTA2, ZSCAN30, |
| | CREM, HMGA2, ZC3H6, ZFPM1, ZNF827, | ZFPM2, RFXANK, IRX6, HMGA2, ZFPM1, |
| | ZNF616, TSHZ3, ZFHX3, CBFB, ZNF514, ARNT2, | MLLT11, ZFHX3, CEBPD, BNC1, ZNF783, |
| | ZBTB4, SMAD7, GATA6, ZNF320, RXRB, EZH1, | MEISI, ANKZF1, PHB, ARNT, GCFC2, ZNF160, |
| | HLX, TOX3, GLIS2, NROB1, ZSCAN18, ETV1, HIC1, | RERE, ZNF85, L3MBTL2, PPARA, ZNF846, |
| | MAFB, ID4, ZP41, THAP6, ZNF644, ZBTB1, DPF3 | RBM22, SALL1, FOXO3, EZH1, CREM, |
| | | ZNF514, ZNF428, THAP6, DPF3, IRF1, AHRR, |
| | | RXRB, ZNF248, ELF1 |
| TCF21 | MEISI, ZNF778, TSHZ3, CUX1, NR1D2, GATA3, | GATA3, MEISI, WTI, HLX, IRF9, PHB, SHZ3, |
| | IRF9, WTI, ZNF616, ZEB2, IRF1, ARID5B, | NROBI, ARID5B, ZFHX4, CREM, NFATC4, |
| | ZBTB20, ZNF827, ETV1, CREM, RXRB, L3MBTL2, | ZNF827, MTA2, ZBTB20, MLLT11, ZNF616, |
| | PHB, NR2C2, ZNF83, AHRR, PHF5A, ARNT, | ETV1, TRPS1, NR2C2, FOSL2, ARNT, |
| | DDIT3, AEBP1, IFI16, MLLT11, ZC3H6, ZNF337, | L3MBTL2, ZNF83, ZEB2, STAT6, AEBP1, |
| | SMAD9, ZNF277, TRPS1, ZNF787, SALL1, ATF4, | NOC4L, ZNF460, EGR2, HIC1, SOX9, ZNF248, |
| | ETV5, ZNF581, ZNF706, JUNB, NFATC4, ZNF639, | AHRR, ZKSCAN1, SMAD9, GABPB2, ETV5, |
| | HIC1, ZKSCAN1, ZNF133, ZFP64, GATA4, ZNF625 | ATRX, HOXB3, NFAT5, CEBPB, ZNF25, JUN, |
| | | ZNF581, ZFPM1, ID2, BCL11A, LRRFIP2, |
| | | ZNF70, ZNF641, JUNB |

### Example 8: BNC1 is Necessary for Epicardial Heterogeneity

To investigate the function of BNC1 in hPSC-epi, we generated hPSCs which were genetically modified with tetracycline (TET)-inducible shRNA for BNC1 knock-down. The cells were treated with TET from the last day of the lateral plate mesoderm stage and during the entire differentiation to hPSC-epi. QPCR, Western-blotting and immunofluorescence showed robust BNC1 silencing under TET treatment (more than 90% by RT-PCR) (FIG. 7A, B, C). BNC1 is necessary for epicardial heterogeneity. We measured the expression of WT1 and TCF21. WT1 was down-regulated four-fold (FIG. 7D) while TCF21 was up-regulated six-fold (FIG. 7F) when the hPSC-epi was differentiated under low level of BNC1. Double-immunostaining against TCF21 and WT1 confirmed that the low-BNC1 hPSC-epi contained a majority of TCF21^{high} cells (FIG. 7E). As expected there was also significant increase in THY1⁺ cell proportions (from 35% to 63%) (FIGS. 7E and G).

We also tested the effect of BNC1 knock-down in human foetal samples by transfecting primary epicardial cultures derived from foetuses over 10 weeks with small interfering RNA. In line with the results obtained in the hPSC-epi, the silencing of BNC1 induced a significant (p=0.02) five-fold increase in in TCF21 expression (FIG. 7H, I, J).

In conclusion in the absence of BNC1, the hPSC-epi behaves as a TCF21^{high} population. Therefore, by suppressing the expression of a single transcription factor, we have modified the cell heterogeneity of the hPSC-epi. Thus we are able to generate a pure TCF21^{high} epicardial population, without requiring sorting methods, as an important step to generate fine-tuned sub-populations of epicardial cells with more specific biological activities.

### Example 8: Epicardial Subtype Isolation

Both the anti-THY1 antibody and anti-PODXL antibody based selection processes are improved further by the use of high stringency depleting columns. Previously, THY1^{low}PODXL^{low} (double-positive) epicardial cells, which express both markers at a low frequency, were present in the negatively selected cell population due to low affinity interactions with the low stringency depleting column. The use of high stringency depleting columns mitigates this, as the double-positive cells are retained in the positively selected population, producing a pure population of negatively selected cells. Where an anti-PODXL antibody is combined with a high-stringency depleting column, a pure population of PODXL-(i.e. THY1+ and TCF21^{high}) cells, absent of antibody activation can be isolated. Where an anti-THY1 antibody is combined with a high-stringency depleting column, a pure population of THY-cells (i.e. PODXL+ and BNC1^{high}), absent of antibody activation can be isolated.

FIG. 10 shows sorting of THY1⁺ cells with LD column and anti-PODXL antibody. The unique specifications of LD columns, given by specific shape and matrix, result in a slower flow rate, as compared with the flow rate of LS columns. Thus, also weakly PODXL⁺ labelled cells are retained with high efficiency and the eluate is purer in THY1⁺ cells. With LS columns, the PODXL⁺ weakly labelled cells do not stay in the column and contaminate the THY1⁺ flow through fraction. These unactivated and better sorted epicardial subtypes will be used for the following experiments which were to be completed but have been delayed due to Covid 19 restrictions.

### Example 9: To Test the Ability of the Two Specified Subpopulations to Support Endothelial Network Formation

We will seed HUVECs together with the sorted epicardial subtypes on Matrigel and measure network formation as previously reported (Bargehr et al. Stem Cells Trans Med 2016). We will also test the ability of the sorted epicardial subtypes to support angiogenesis in a chick chorioallantoic membrane assay (Bargehr et al Nature Biotech 2019).

We expect that different subpopulations of epicardial cells will have different effects on endothelial network formation, survival and mural cell recruitment, showing the utility of the separation protocol for developing better cell therapy reagents.

Experiment 2. To Determine the Ability of Sorted Epicardial Subtypes to Improve Cardiomyocyte Function in 3D-Engineered Heart Tissues (3D-EHT) In Vitro.

Using our previously described methods (Bargehr et al Nature Biotech 2019) we will seed human pluripotent stem cell derived cardiomyocytes in a collagen gel together with epicardial cell subtypes. The constructs will be allowed to mature and then measured for contraction, Ca2+ flux, cardiomyocyte maturity and electrical integration.

1. A method for separating in vitro-differentiated human epicardial cells into a first population of cells characterised by higher levels of expression of transcription factor 21 when compared to basonuclin 1 and a second population of cells characterised by higher levels of expression basonuclin 1 when compared to transcription factor 21, the method comprising cell-sorting based upon the use of a capture agent specific to a cell surface marker for the first population of cells and/or the second population of cells.

2. A method for separating in vitro-differentiated human epicardial cells according to claim 1, wherein said cells are derived from human induced pluripotent stem cells.

3. A method for separating in vitro-differentiated human epicardial cells according to claim 1 or claim 2, wherein the first population of cells expresses at least 5, at least 8, at least 10 times more transcription factor 21 than basonuclin 1.

4. A method for separating in vitro-differentiated human epicardial cells according to any one of the preceding claims, wherein the cell surface protein marker for the first population of cells is selected from the group consisting of THY1, SIPR3, PDGFRA, BAMBI, PLD3, ADAM12, TGFBR3, STRA6, SLC12A8, BEST1, SMIM3, NRP1, ITGA1, TEK, IGDCC4, CD99, ABCA1, CD9, NDRG2, IFITM1 and ACVR2A.

5. A method for separating in vitro-differentiated human epicardial cells according to claim 4, wherein the cell surface protein marker for the first population of human epicardial cells is THY1.

6. A method for separating in vitro-differentiated human epicardial cells according to claim 5, wherein the capture agent is an antibody, preferably a mouse anti-THY1 antibody.

7. A method for separating in vitro-differentiated human epicardial cells according to any one of claims 1 to 3, wherein the cell surface marker for the second population is selected from the group consisting of PODXL, LRP2, ITGA6, TMEM98, CDH3, CDH1, LEPROTL1, SLC34A2, PKHD1L1, AQP1, GPNMB, SLC7A7, CNTN6, CXADR, SLC4A8, PTPRF, ATP7B, ACKR3, SLC2A1, SLC16A3, OLR1, TMEM88, S100A10, CD82, PARM1, PLXNB2 and APLP2.

8. A method for separating in vitro-differentiated human epicardial cells according to claim 7, wherein the cell surface marker for the second population of human epicardial cells is PODXL.

9. A method for separating in vitro-differentiated human epicardial cells according to any one of the preceding claims, said method comprising cell-sorting based upon the use of a capture agent specific to a cell surface marker for the first population of cells and/or the second population of cells and collecting the eluted cells.

10. A method for separating in vitro-differentiated human epicardial cells according to any one of the preceding claims, wherein under cardiac fibroblast differentiation conditions more cells in the first population differentiate forming cardiac fibroblasts than cells in the second population.

11. A method for separating in vitro-differentiated human epicardial cells according to claim 8 or 9, wherein more than 50, more than 60, more than 70, more than 80% of the cells in the first population differentiate forming cardiac fibroblasts.

12. A method for separating in vitro-differentiated human epicardial cells according to claim 8, 9 or claim 10, wherein less than 50, less than 40, less than 30, less than 20% of the cells in the second population differentiate forming cardiac fibroblasts.

13. An isolated first population of in vitro-differentiated human epicardial cells characterised by higher expression of transcription factor 21 when compared to basonuclin 1.

14. An isolated first population of in vitro-differentiated human epicardial cells according to claim 13, wherein the first population expresses at least 5, at least 8, at least 10 times more transcription factor 21 than basonuclin 1.

15. An isolated first population of in vitro-differentiated human epicardial cells according to claim 13 or claim 14, wherein under cardiac fibroblast differentiation conditions more than 50, more than 60, more than 70, more than 80% of the cells in the first population of human epicardial cells differentiate forming cardiac fibroblasts.

16. An isolated second population of in vitro-differentiated human epicardial cells characterised by higher expression of basonuclin 1 when compared to transcription factor 21.

17. An isolated second population of in vitro-differentiated human epicardial cells according to claim 16, wherein under cardiac fibroblast differentiation conditions less than 50, less than 40, less than 30, less than 20% of the cells in the second population differentiate forming cardiac fibroblasts.

18. A mixture of first and second populations of in vitro-differentiated human epicardial cells, wherein the first population is characterised by higher expression of transcription factor 21 when compared to basonuclin 1 and the second population is characterised by higher expression of basonuclin 1 when compared to transcription factor 21, wherein the mixture is enriched with either the first or second populations.

19. A mixture according to claim 18, wherein the mixture is formed by the combination of unseparated in vitro-differentiated human epicardial cells and either the first or second populations after separation.

20. A mixture according to claim 18 or claim 19 additionally comprising cardiomyocytes.

21. A mixture of first and second populations of in vitro-differentiated human epicardial cells according to claim 18, 19 or claim 20, wherein the first population expresses at least 5, at least 8, at least 10 times more transcription factor 21 than basonuclin 1.

22. A mixture of first and second populations of in vitro-differentiated human epicardial cells according to any one of claims 18 to 21, wherein under cardiac fibroblast differentiation conditions more than 50, more than 60, more than 70, more than 80% of the cells in the first population differentiate forming cardiac fibroblasts.

23. A mixture of first and second populations of in vitro-differentiated human epicardial cells according to any one of claims 18 to 21, wherein under cardiac fibroblast differentiation conditions less than 50, less than 40, less than 30, less than 20% of the cells in the second population differentiate forming cardiac fibroblasts.

24. An isolated first population of in vitro-differentiated human epicardial cells according to any one of claims 13 to 15 or an isolated second population of in vitro-differentiated human epicardial cells according to claim 16 or claim 17 or a mixture of first and second populations of in vitro-differentiated human epicardial cells according to any one of claims 18 to 22 for use as a medicament.

25. An isolated first population of in vitro-differentiated human epicardial cells according to any one of claims 13 to 15 or an isolated second population of in vitro-differentiated human epicardial cells according to claim 16 or claim 17 or a mixture of first and second populations of in vitro-differentiated human epicardial cells according to any one of claims 18 to 23 for use in treating and/or repairing cardiac tissue damage.

26. An isolated first population of in vitro-differentiated human epicardial cells according to any one of claims 12 to 14 or a mixture of first and second populations of in vitro-differentiated human epicardial cells according to any one of claims 17 to 21 wherein the mixture is enriched with the first population for use in treating and/or repairing cardiac blood vessel, smooth muscle fibre or cardiac fibroblast damage.

27. An isolated second population of in vitro-differentiated human epicardial cells according to claim 15 or claim 16 or a mixture of first and second populations of in vitro-differentiated human epicardial cells according to any one of claims 17 to 21 wherein the mixture is enriched with the second population for use in treating and/or repairing smooth muscle fibre damage, preferably with reduced fibrosis.

28. A pharmaceutical composition comprising the invitro—differentiated cell population of claims 13 to 15 or an isolated second population of in vitro-differentiated human epicardial cells according to claim 16 or claim 17 or a mixture of first and second populations of in vitro-differentiated human epicardial cells according to any one of claims 18 to 23 and a pharmaceutically acceptable excipient.

29. A method for the production of a first population of in vitro-differentiated epicardial cells, characterised by higher levels of expression of transcription factor 21 when compared to basonuclin 1, comprising the knockdown of BNC1 in human pluripotent stem cells.


SEQUENCE LISTING (10 sequences)

SEQ ID NO: 1 - DNA, 21 bp, Artificial Sequence
  AAVsingiKD forward primer
cgaacgctgacgtcatcaacc

SEQ ID NO: 2 - DNA, 22 bp, Artificial Sequence
  AAVsingiKD reverse primer
gggctatgaactaatgaccccg

SEQ ID NO: 3 - DNA, 21 bp, Artificial Sequence
  GAPDH forward primer
aacagcctcaagatcatcagc

SEQ ID NO: 4 - DNA, 20 bp, Artificial Sequence
  GAPDH reverse primer
ggatgatgttctggagagcc

SEQ ID NO: 5 - DNA, 20 bp, Artificial Sequence
  WT1 forward primer
cacagcacagggtacgagag

SEQ ID NO: 6 - DNA, 20 bp, Artificial Sequence
  WT1 reverse primer
caagagtcggggctactcca

SEQ ID NO: 7 - DNA, 22 bp, Artificial Sequence
  TCF21 forward primer
tcctggctaacgacaaatacga

SEQ ID NO: 8 - DNA, 20 bp, Artificial Sequence
  TCF21 reverse primer
tttcccggccaccataaagg

SEQ ID NO: 9 - DNA, 22 bp, Artificial Sequence
  BNC1 forward primer
ggccgaggctatcagctgtact

SEQ ID NO: 10 - DNA, 20 bp, Artificial Sequence
  BNC1 reverse primer
gcctgggtcccatagagcat